(19) United States
(12) Patent Application Publication
(10) Pub. No.: US 2023/0220424 A1
(43) Pub. Date: Jul. 13, 2023

(54) RAPID REMOVAL OF A SELF-REPLICATING FUNGAL PLASMID FOR EFFICIENT MARKER CYCLING

(71) Applicant: Zymergen Inc., Emeryville, CA (US)

(72) Inventors: Elise DE REUS, Emeryville, CA (US); Kenneth S. BRUNO, Walnut Creek, CA (US); Gareth BUTLAND, Danville, CA (US)

(21) Appl. No.: 18/155,552

(22) Filed: Jan. 17, 2023

Related U.S. Application Data
(63) Continuation of application No. PCT/US2021/041787, filed on Jul. 15, 2021.
(60) Provisional application No. 63/053,069, filed on Jul. 17, 2020.

(51) Int. Cl. C12N 15/90; C12N 15/11; C12N 9/22; C12N 15/80

(52) U.S. Cl. CPC C12N 15/905; C12N 15/11; C12N 9/22; C12N 15/80; C12N 2310/20; C12N 2800/80; C12N 2830/002; C12N 2800/102

(57) ABSTRACT

The present disclosure provides compositions and methods for gene editing. The disclosure also provides methods for removing extra-chromosomally replicating plasmids from competent cells.

### CROSS-REFERENCE TO RELATED APPLICATIONS

This application is a continuation of International Patent Application No. PCT/US2021/041787, filed Jul. 15, 2021, which claims priority to and the benefit of U.S. Provisional Patent Application No. 63/053,069, filed Jul. 17, 2020, which is incorporated by reference herein in its entirety.

### REFERENCE TO AN ELECTRONIC SEQUENCE LISTING

The contents of the electronic sequence listing (ZYMR_074_01 US_SeqList_ST26.xml; Size: 13,565 bytes; and Date of Creation: Jan. 17, 2023) are herein incorporated by reference in its entirety.

### FIELD

The present disclosure generally describes methods of removing extra-chromosomally replicating plasmids from competent cells. The disclosure further provides methods and compositions for gene editing.

### BACKGROUND

Filamentous fungi are capable of expressing native and heterologous proteins to high levels, making them well-suited for the large-scale production of enzymes, proteins, small molecules, and natural products for industrial, pharmaceutical, animal health, and food and beverage applications. The use of filamentous fungi for large-scale production of products of interest requires genetic manipulation of the selected fungi for improving strain performance in industrial applications.

Extra-chromosomally replicating plasmids are a valuable tool for genome editing of filamentous fungi, because they enable genome editing without integration of a selectable marker gene, so called ‘marker-free’ genome editing. However, the difficulty of removing extra-chromosomally replicating plasmids from the filamentous fungi prevents these plasmids from being used or recycled for successive rounds of genome editing. Known methods for plasmid removal include culturing the fungi in counter-selective conditions or without selective pressure or subjecting the fungus to asexual sporulation to achieve clonality. These methods are time-consuming, may require chemicals that are mutagenic to the microbial host or toxic to humans, and are especially challenging for fungal strains that do not sporulate. Thus, there is a need in the art for new methods of recycling extra-chromosomally replicating plasmids.

### SUMMARY

The present disclosure solves the problems in the art by providing compositions and methods for efficient genome editing in filamentous fungi, which are markerless. The present disclosure provides novel methods and compositions for gene editing. The compositions of the disclosure comprise an extra-chromosomally replicating plasmid comprising a selectable marker gene, a gene-editing complex that recognizes a genomic target in a competent cell, and a reagent for the removal of the extra-chromosomally replicating plasmid. The compositions optionally comprise a genetic element of interest. The disclosure further provides methods for using these compositions to recycle an extra-chromosomally replicating plasmid and for making multiple edits to the genome of a filamentous fungi.

In some embodiments, provided herein are compositions for gene editing, comprising: competent cells; an extra-chromosomally replicating plasmid comprising a selectable marker gene; and a gene-editing complex that recognizes a genomic target of a competent cell.

In some embodiments, the compositions comprise a genetic element of interest.

In some embodiments, the compositions do not comprise a genetic element of interest.

In some embodiments, the genetic element of interest is selected from the group consisting of: a nucleic acid sequence, a gene of interest, a gene variant, a genetic edit, a single nucleotide polymorphism, a genetic regulatory sequence, a promoter, a non-coding nucleic acid sequence, a terminator, or any combination thereof.

In some embodiments, the genetic element of interest is a promoter.

In some embodiments, the genetic element of interest is a gene or fragment thereof.

In some embodiments, the gene-editing complex comprises a ribonucleoprotein (RNP).

In some embodiments, the RNP comprises Cas9 and a guide RNA (gRNA) that recognizes the genomic target.

In some embodiments, the gene-editing complex comprises a transcription activator-like effector nuclease (TALEN).

In some embodiments, the gene-editing complex comprises a zinc-finger nuclease (ZFN).

In some embodiments, the competent cells are eukaryotic cells.

In some embodiments, the competent cells are prokaryotic cells.

In some embodiments, the competent cells are fungal cells.

In some embodiments, the competent cells are filamentous fungal cells.

In some embodiments, the competent cells are protoplasts.

In some embodiments, the extra-chromosomally replicating plasmid comprises a plasmid replicator.

In some embodiments, the plasmid replicator is AMA1.

In some embodiments, the selectable marker gene is selected from pvrG, hph, nat, amdS, nptII, niaD, and argB.

In some embodiments, the extra-chromosomally replicating plasmid comprises an endonuclease site.

In some embodiments, the extra-chromosomally replicating plasmid comprises a recombinatorial site.

In some embodiments, the recombinatorial site is a loxP site or a Frt site.

In some embodiments, the compositions comprise a RNP that recognizes the selectable marker gene.

In some embodiments, the RNP comprises Cas9 and a gRNA.

In some embodiments, the compositions comprise an endonuclease, which recognizes an endonuclease site.

In some embodiments, the compositions comprise a recombinase, which recognizes a recombinatorial site.

In some embodiments, the extra-chromosomally replicating plasmid comprises a suicide gene, wherein the suicide gene is under control of an inducible promoter.

In some embodiments, the inducible promoter is an alcohol-regulated promoter, a tetracycline-regulated promoter, a steroid regulated promoter, a metal-regulated promoter, a pathogenesis regulated promoter, a carbon-regulated promoter, a xylose-regulated promoter, a heat shock promoter, a synthetic-transcription factor-dependent promoter, or a light-regulated promoter.

In some embodiments, expression of the suicide gene is induced by an alcohol, a transcription factor, tetracycline, a steroid, a metal, heat, light, an antibiotic, a sugar, xylose, glucose, sucrose, maltose, ethanol, glycerol, methanol, oleic acid, acetate, hexose, lactose, or galactose.

In some embodiments, provided herein is a method for gene editing, comprising: transforming a competent cell with a first composition comprising: an extra-chromosomally replicating plasmid comprising a selectable marker gene and a gene-editing complex that recognizes a genomic target of a competent cell.

In some embodiments, the first composition comprises a genetic element of interest.

In some embodiments, the first composition does not comprise a genetic element of interest.

In some embodiments, the genetic element of interest of the first composition is selected from the group consisting of: a nucleic acid sequence, a gene of interest, a gene variant, a genetic edit, a single nucleotide polymorphism, a genetic regulatory sequence, a promoter, a non-coding nucleic acid sequence, a terminator, or any combination thereof.

In some embodiments, the genetic element of interest of the first composition is a promoter.

In some embodiments, the genetic element of interest of the first composition is a gene or fragment thereof.

In some embodiments, the gene-editing complex of the first composition comprises a ribonucleoprotein (RNP).

In some embodiments, the gene-editing complex of the first composition comprises a ribonucleoprotein (RNP), wherein the RNP comprises Cas9 and a guide RNA (gRNA) that recognizes the genomic target.

In some embodiments, the gene-editing complex of the first composition comprises a transcription activator-like effector nuclease (TALEN).

In some embodiments, the gene-editing complex of the first composition comprises a zinc-finger nuclease (ZFN).

In some embodiments, the methods of the disclosure comprise selecting for competent cells that comprise the extra-chromosomally replicating plasmid.

In some embodiments, the extra-chromosomally replicating plasmid of the first composition comprises a plasmid replicator.

In some embodiments, the extra-chromosomally replicating plasmid of the first composition comprises a plasmid replicator, wherein the plasmid replicator is AMA1.

In some embodiments, the selectable marker gene of the extra-chromosomally replicating plasmid of the first composition is selected from pyrG, hph, nat, amdS, nptII, niaD, and argB.

In some embodiments, the extra-chromosomally replicating plasmid of the first composition comprises a endonuclease site.

In some embodiments, the extra-chromosomally replicating plasmid of the first composition comprises a recombinatorial site.

In some embodiments, the extra-chromosomally replicating plasmid of the first composition comprises a recombinatorial site, wherein the recombinatorial site is a loxP site or a Frt site.

In some embodiments, the methods of the disclosure comprise removing the extra-chromosomally replicating plasmid.

In some embodiments, the methods of the disclosure comprise removing the extra-chromosomally replicating plasmid by applying a RNP to the competent cells comprising the extra-chromosomally replicating plasmid.

In some embodiments, the methods comprise removing the extra-chromosomally replicating plasmid by applying a RNP to the competent cells comprising the extra-chromosomally replicating plasmid, wherein the RNP comprises Cas9 and a gRNA that recognizes the selectable marker gene of the extra-chromosomally replicating plasmid.

In some embodiments, the methods comprise removing the extra-chromosomally replicating plasmid by applying a recombinase to the competent cells comprising the extra-chromosomally replicating plasmid, wherein the recombinase recognizes a recombinatorial site on the extra-chromosomally replicating plasmid.

In some embodiments, the methods comprise removing the extra-chromosomally replicating plasmid by applying an endonuclease to the competent cells comprising the extra-chromosomally replicating plasmid, wherein the endonuclease recognizes an endonuclease site on the extra-chromosomally replicating plasmid.

In some embodiments, the methods comprise introducing a genetic element of interest at a genomic target site.

In some embodiments, the extra-chromosomally replicating plasmid of the first composition comprises a suicide gene, wherein the suicide gene is under control of an inducible promoter.

In some embodiments, the extra-chromosomally replicating plasmid of the first composition comprises a suicide gene, wherein the suicide gene is under control of an inducible promoter, wherein the inducible promoter is an alcohol-regulated promoter, a tetracycline-regulated promoter, a steroid regulated promoter, a metal-regulated promoter, a pathogenesis regulated promoter, a heat shock promoter, a carbon-regulated promoter, a xylose-regulated promoter, a synthetic-transcription factor-dependent promoter or a light-regulated promoter.

In some embodiments, the methods comprise removing the extra-chromosomally replicating plasmid by inducing the promoter to express the suicide gene.

In some embodiments, inducing comprises introducing an alcohol, a transcription factor, tetracycline, a steroid, a metal, heat, light, an antibiotic, a sugar, xylose, glucose, sucrose, maltose, ethanol, glycerol, methanol, oleic acid, acetate, hexose, lactose, or galactose to the competent cells comprising the extra-chromosomally replicating plasmid.

In some embodiments, the methods comprise introducing a gene-editing complex of the first composition that recognizes a genomic target of a competent cell, wherein the competent cell is a eukaryotic cell.

In some embodiments, the methods comprise introducing a gene-editing complex of the first composition that recognizes a genomic target of a competent cell, wherein the competent cell is a prokaryotic cell.

In some embodiments, the methods comprise introducing a gene-editing complex of the first composition that recognizes a genomic target of a competent cell, wherein the competent cell is a fungal cell.

In some embodiments, the methods comprise introducing a gene-editing complex of the first composition that recognizes a genomic target of a competent cell, wherein the competent cell is a filamentous fungal cell.

In some embodiments, the methods comprise introducing a gene-editing complex of the first composition that recognizes a genomic target of a competent cell, wherein the competent cell is a protoplast.

In some embodiments, the methods comprise introducing a second composition, comprising: a second extra-chromosomally replicating plasmid, wherein the extra-chromosomally replicating plasmid comprises a second selectable marker gene and a gene-editing complex that recognizes a genomic target of a competent cell.

In some embodiments, the second composition comprises a genetic element of interest.

In some embodiments, the second composition does not comprise a genetic element of interest.

In some embodiments, the genetic element of interest of the second composition is selected from the group consisting of: a nucleic acid sequence, a gene of interest, a gene variant, a genetic edit, a single nucleotide polymorphism, a genetic regulatory sequence, a promoter, a non-coding nucleic acid sequence, a terminator, or any combination thereof.

In some embodiments, the genetic element of interest of the second composition is a promoter.

In some embodiments, the genetic element of interest of the second composition is a gene or fragment thereof.

In some embodiments, the gene-editing complex of the second composition comprises a ribonucleoprotein (RNP).

In some embodiments, the gene-editing complex of the second composition comprises a ribonucleoprotein (RNP), wherein the RNP comprises Cas9 and a guide RNA (gRNA) that recognizes the genomic target.

In some embodiments, the gene-editing complex of the second composition comprises a transcription activator-like effector nuclease (TALEN).

In some embodiments, the gene-editing complex of the second composition comprises a zinc-finger nuclease (ZFN).

In some embodiments, the methods comprise selecting for competent cells that comprise the second extra-chromosomally replicating plasmid.

In some embodiments, the second extra-chromosomally replicating plasmid comprises a plasmid replicator.

In some embodiments, the second extra-chromosomally replicating plasmid comprises a plasmid replicator, wherein the plasmid replicator is AMA1.

In some embodiments, the second selectable marker gene is selected from pyrG, hph, nat, amdS, nptII, niaD, and argB.

In some embodiments, the second extra-chromosomally replicating plasmid comprises an endonuclease site.

In some embodiments, the second extra-chromosomally replicating plasmid comprises a recombinatorial site.

In some embodiments, the second extra-chromosomally replicating plasmid comprises a recombinatorial site, wherein the recombinatorial site is a loxP site or a Frt site.

In some embodiments, the methods comprise removing the second extra-chromosomally replicating plasmid.

In some embodiments, the methods comprise removing the second extra-chromosomally replicating plasmid by applying a ribonucleoprotein (RNP) to the competent cells comprising the second extra-chromosomally replicating plasmid.

In some embodiments, the methods comprise removing the second extra-chromosomally replicating plasmid by applying a ribonucleoprotein (RNP) to the competent cells comprising the second extra-chromosomally replicating plasmid, wherein the RNP comprises Cas9 and a gRNA that recognizes the selectable marker gene of the second extra-chromosomally replicating plasmid.

In some embodiments, the methods comprise removing the second extra-chromosomally replicating plasmid by applying a recombinase to the competent cells comprising the second extra-chromosomally replicating plasmid, wherein the recombinase recognizes a recombinatorial site on the second extra-chromosomally replicating plasmid.

In some embodiments, the methods comprise removing the second extra-chromosomally replicating plasmid by applying an endonuclease to the competent cells comprising the extra-chromosomally replicating plasmid, wherein the endonuclease recognizes an endonuclease site on the extra-chromosomally replicating plasmid.

In some embodiments, the genetic element of interest of the second composition is introduced at a genomic target site of the second composition.

In some embodiments, the extra-chromosomally replicating plasmid of the second composition comprises a suicide gene, wherein the suicide gene is under control of an inducible promoter.

In some embodiments, the extra-chromosomally replicating plasmid of the second composition comprises a suicide gene, wherein the suicide gene is under control of an inducible promoter, wherein the inducible promoter is an alcohol-regulated promoter, a tetracycline-regulated promoter, a steroid regulated promoter, a metal-regulated promoter, a pathogenesis regulated promoter, a heat shock promoter, a carbon-regulated promoter, a xylose-regulated promoter, a synthetic-transcription factor-dependent promoter, or a light-regulated promoter.

In some embodiments, the methods comprise removing the extra-chromosomally replicating plasmid of the second composition by inducing the promoter which controls the suicide gene to express the suicide gene.

In some embodiments, the methods comprise removing the extra-chromosomally replicating plasmid of the second composition by inducing the promoter which controls the suicide gene to express the suicide gene, wherein inducing comprises introducing an alcohol, a transcription factor, tetracycline, a steroid, a metal, heat, light, an antibiotic, a sugar, xylose, glucose, sucrose, maltose, ethanol, glycerol, methanol, oleic acid, acetate, hexose, lactose, or galactose to the competent cells comprising the second extra-chromosomally replicating plasmid.

In some embodiments, the methods comprise a gene-editing complex of the second composition that recognizes a genomic target of a competent cell, wherein the competent cell is a eukaryotic cell.

In some embodiments, the methods comprise introducing a gene-editing complex of the second composition that recognizes a genomic target of a competent cell, wherein the competent cell is a prokaryotic cell.

In some embodiments, the methods comprise introducing a gene-editing complex of the second composition that recognizes a genomic target of a competent cell, wherein the competent cell is a fungal cell.

In some embodiments, the methods comprise introducing a gene-editing complex of the second composition that recognizes a genomic target of a competent cell, wherein the competent cell is a filamentous fungal cell.

In some embodiments, the methods comprise introducing a gene-editing complex of the second composition that recognizes a genomic target of a competent cell, wherein the competent cell is a protoplast.

In some embodiments, the disclosure provides a method of removing an extra-chromosomally replicating plasmid comprising a selectable marker gene from a competent cell, comprising: administering a reagent to remove the extra-chromosomally replicating plasmid.

In some embodiments, the disclosure provides a method of removing an extra-chromosomally replicating plasmid comprising a selectable marker gene from a competent cell, comprising: administering a reagent to remove the extra-chromosomally replicating plasmid, wherein the extra-chromosomally replicating plasmid comprises a plasmid replicator.

In some embodiments, the disclosure provides a method of removing an extra-chromosomally replicating plasmid comprising a selectable marker gene from a competent cell, comprising: administering a reagent to remove the extra-chromosomally replicating plasmid, wherein the extra-chromosomally replicating plasmid comprises a plasmid replicator, wherein the plasmid replicator is AMA1.

In some embodiments, the disclosure provides a method of removing an extra-chromosomally replicating plasmid comprising a selectable marker gene from a competent cell, wherein the selectable marker gene is selected from pyrG, hph, nat, amdS, nptII, niaD, and argB.

In some embodiments, the disclosure provides a method of removing an extra-chromosomally replicating plasmid comprising a selectable marker gene from a competent cell, wherein the competent cell is a eukaryotic cell.

In some embodiments, the disclosure provides a method of removing an extra-chromosomally replicating plasmid comprising a selectable marker gene from a competent cell, wherein the competent cell is a prokaryotic cell.

In some embodiments, the disclosure provides a method of removing an extra-chromosomally replicating plasmid comprising a selectable marker gene from a competent cell, wherein the competent cell is a fungal cell.

In some embodiments, the disclosure provides a method of removing an extra-chromosomally replicating plasmid comprising a selectable marker gene from a competent cell, wherein the competent cell is a filamentous fungal cell.

In some embodiments, the disclosure provides a method of removing an extra-chromosomally replicating plasmid comprising a selectable marker gene from a competent cell, wherein the competent cell is a protoplast.

In some embodiments, the disclosure provides a method of removing an extra-chromosomally replicating plasmid comprising a selectable marker gene from a competent cell, comprising: administering a reagent to remove the extra-chromosomally replicating plasmid, wherein the reagent comprises a ribonucleoprotein (RNP), an endonuclease, or a recombinase.

In some embodiments, the disclosure provides a method of removing an extra-chromosomally replicating plasmid comprising a selectable marker gene from a competent cell, comprising: administering a reagent to remove the extra-chromosomally replicating plasmid, wherein the reagent comprises a ribonucleoprotein (RNP) that recognizes the selectable marker gene.

In some embodiments, the disclosure provides a method of removing an extra-chromosomally replicating plasmid comprising a selectable marker gene from a competent cell, comprising: administering a reagent to remove the extra-chromosomally replicating plasmid, wherein the reagent comprises a ribonucleoprotein (RNP) that recognizes the selectable marker gene, wherein the RNP comprises Cas9 and a gRNA.

In some embodiments, the disclosure provides a method of removing an extra-chromosomally replicating plasmid, wherein the extra-chromosomally replicating plasmid comprises an endonuclease site.

In some embodiments, the disclosure provides a method of removing an extra-chromosomally replicating plasmid comprising a selectable marker gene from a competent cell, comprising: administering a reagent to remove the extra-chromosomally replicating plasmid, wherein the reagent is an endonuclease that recognizes an endonuclease site.

In some embodiments, the disclosure provides a method of removing an extra-chromosomally replicating plasmid comprising a selectable marker gene from a competent cell, wherein the extra-chromosomally replicating plasmid comprises a recombinatorial site.

In some embodiments, the disclosure provides a method of removing an extra-chromosomally replicating plasmid comprising a selectable marker gene from a competent cell, wherein the extra-chromosomally replicating plasmid comprises a recombinatorial site, wherein the recombinatorial site is a loxP site or a Frt site.

In some embodiments, the disclosure provides a method of removing an extra-chromosomally replicating plasmid comprising a selectable marker gene from a competent cell, comprising administering a reagent to remove the extra-chromosomally replicating plasmid, wherein the reagent comprises a recombinase that recognizes a recombinatorial site.

In some embodiments, the disclosure provides a method of removing an extra-chromosomally replicating plasmid, wherein the extra-chromosomally replicating plasmid comprises a suicide gene, wherein the suicide gene is under control of an inducible promoter.

In some embodiments, the disclosure provides a method of removing an extra-chromosomally replicating plasmid, wherein the extra-chromosomally replicating plasmid comprises a suicide gene, wherein the suicide gene is under control of an inducible promoter, wherein the inducible promoter is an alcohol-regulated promoter, a tetracycline-regulated promoter, a steroid regulated promoter, a metal-regulated promoter, a pathogenesis regulated promoter, a heat shock promoter, a carbon-regulated promoter, a xylose-regulated promoter, a synthetic-transcription factor-dependent promoter, or a light-regulated promoter.

In some embodiments, the disclosure provides a method of removing an extra-chromosomally replicating plasmid, wherein the extra-chromosomally replicating plasmid comprises a suicide gene, wherein the suicide gene is under control of an inducible promoter, wherein the inducible promoter is an alcohol-regulated promoter, a tetracycline-regulated promoter, a steroid regulated promoter, a metal-regulated promoter, a pathogenesis regulated promoter, a heat shock promoter, a carbon-regulated promoter, a xylose-regulated promoter, a synthetic-transcription factor-dependent promoter, or a light-regulated promoter, comprising introducing a reagent to induce expression of the suicide gene, wherein the reagent is selected from the group consisting of a metal, a transcription factor, heat, light, an antibiotic, a sugar, xylose, glucose, sucrose, maltose, ethanol, glycerol, methanol, oleic acid, acetate, hexose, lactose, and galactose.

In some embodiments, the disclosure provides a method for making markerless multiple genomic edits, comprising:

(a) transforming a competent cell with a first composition comprising:
(i) an extra-chromosomally replicating plasmid comprising a selectable marker gene; and
(ii) a gene-editing complex that recognizes a genomic target of a competent cell;

(b) selecting for competent cells that comprise the extra-chromosomally replicating plasmid of the first composition;

(c) removing the extra-chromosomally replicating plasmid of the first composition by administering a reagent;

(d) transforming a competent cell with a second composition comprising:
(i) an extra-chromosomally replicating plasmid comprising a selectable marker gene; and
(ii) a gene-editing complex that recognizes a genomic target of a competent cell;

(e) selecting for competent cells that comprise the extra-chromosomally replicating plasmid of the second composition; and

(f) removing the extra-chromosomally replicating plasmid of the second composition by administering a reagent.

In some embodiments, the first composition comprises a genetic element of interest.

In some embodiments, the second composition comprises a genetic element of interest.

In some embodiments, the disclosure provides a method for making markerless multiple genomic edits, comprising:

(a) transforming a competent cell with a first composition comprising:
(i) an extra-chromosomally replicating plasmid comprising a selectable marker gene; and
(ii) a gene-editing complex that recognizes a genomic target of a competent cell;

(b) selecting for competent cells that comprise the extra-chromosomally replicating plasmid of the first composition;

(c) removing the extra-chromosomally replicating plasmid of the first composition by administering a reagent;

(d) transforming a competent cell with a second composition comprising:
(i) an extra-chromosomally replicating plasmid comprising a selectable marker gene; and
(ii) a gene-editing complex that recognizes a genomic target of a competent cell;

(e) selecting for competent cells that comprise the extra-chromosomally replicating plasmid of the second composition; and

(f) removing the extra-chromosomally replicating plasmid of the second composition by administering a reagent

(g) transforming the competent cell with a third composition comprising:
(i) an extra-chromosomally replicating plasmid comprising a selectable marker gene; and
(ii) a gene-editing complex that recognizes a genomic target of a competent cell.

(h) selecting for competent cells that comprise the extra-chromosomally replicating plasmid of the third composition; and
(i) removing the extra-chromosomally replicating plasmid of the third composition by administering a reagent.

In some embodiments, the first extra-chromosomally replicating plasmid is removed by administering a recombinase that recognizes a recombinatorial site on the first extra-chromosomally replicating plasmid.

In some embodiments, the second extra-chromosomally replicating plasmid is removed by administering a recombinase that recognizes a recombinatorial site on the second extra-chromosomally replicating plasmid.

In some embodiments, the first extra-chromosomally replicating plasmid is removed by administering an endonuclease that recognizes an endonuclease site on the first extra-chromosomally replicating plasmid.

In some embodiments, the second extra-chromosomally replicating plasmid is removed by administering an endonuclease that recognizes an endonuclease site on the second extra-chromosomally replicating plasmid.

In some embodiments, the first extra-chromosomally replicating plasmid is removed by administering a RNP that recognizes a selectable marker gene on the first extra-chromosomally replicating plasmid.

In some embodiments, the second extra-chromosomally replicating plasmid is removed by administering a RNP that recognizes a selectable marker gene on the second extra-chromosomally replicating plasmid.

In some embodiments, the RNP comprises a gRNA and Cas9.

In some embodiments, the RNP comprises a gRNA and Cas9.

In some embodiments, the first extra-chromosomally replicating plasmid is removed by administering an inducer of a suicide gene on the first extra-chromosomally replicating plasmid.

In some embodiments, the second extra-chromosomally replicating plasmid is removed by administering an inducer of a suicide gene on the second extra-chromosomally replicating plasmid.

### BRIEF DESCRIPTION OF THE DRAWINGS

FIG. 1 shows a method for gene editing comprising transforming competent cells with a composition comprising an extra-chromosomally-replicating plasmid: competent cells; a gene-editing complex, comprising a ribonucleoprotein (RNP) that recognizes a genomic target; and a genetic element of interest. The extra-chromosomally replicating plasmid of round 1 is removed in round 2 by introducing an RNP that targets marker X (RNP X). Similarly, the extra-chromosomally replicating plasmid of round 2 is removed in round 3 by introducing an RNP that targets marker Y (RNP Y).

FIG. 2 shows a method for gene editing comprising transforming competent cells with a composition comprising an extra-chromosomally-replicating plasmid: competent cells: a gene-editing complex, comprising a ribonucleoprotein (RNP) that recognizes a genomic target; and a genetic element of interest. The extra-chromosomally replicating plasmid of round 1 is removed in round 2 by introducing a recombinase that targets a set of recombinatorial sites, (e.g. motif X). One recombinatorial site is upstream of the selectable marker gene, and one recombinatorial site is downstream of the selectable marker gene. Similarly, the extra-chromosomally replicating plasmid of round 2 is removed in round 3 by introducing a recombinase that targets a second set of recombinatorial sites (e.g. motif Y). One recombinatorial site is upstream of the selectable marker gene, and one recombinatorial site is downstream of the selectable marker gene.

FIG. 3 shows a method for gene editing comprising transforming competent cells with a composition comprising an extra-chromosomally-replicating plasmid; competent cells; a gene-editing complex, comprising a ribonucleoprotein (RNP) that recognizes a genomic target; and a genetic element of interest. The extra-chromosomally replicating plasmid of round 1 is removed in round 2 by introducing an endonuclease that targets an endonuclease site (e.g. motif X). Similarly, the extra-chromosomally replicating plasmid of round 2 is removed in round 3 by introducing a second endonuclease that targets a second endonuclease site (e.g. motif Y).

### DETAILED DESCRIPTION

### I. Definitions

The term “a” or “an” refers to one or more of that entity. i.e., can refer to a plural referent. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A): in yet another embodiment, to both A and B (optionally including other elements); etc.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% (i.e., within 10%, 9%, 8%, 7%, 6%, 5%, 4%, 3%, 2%, 1%, or less) above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

Herein, the terms “comprise”, or variations such as “comprises” or “comprising”, will be understood to imply the inclusion of a stated element or integer or group of elements or integers but not the exclusion of any other element or integer or group of elements or integers.

A “eukaryote” is any organism whose cells contain a nucleus and other organelles enclosed within membranes. Eukaryotes belong to the taxon Eukarya or Eukaryota. The defining feature that sets eukaryotic cells apart from prokaryotic cells (the aforementioned Bacteria and Archaea) is that they have membrane-bound organelles, especially the nucleus, which contains the genetic material, and is enclosed by the nuclear envelope.

“Bacteria” or “eubacteria” refers to a domain of prokaryotic organisms. Bacteria include at least 11 distinct groups as follows: (1) Gram-positive (gram+) bacteria, of which there are two major subdivisions: (i) high G+C group (Actinomycetes, Mycobacteria, Micrococcus, others) (ii) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia: (8) Green sulfur bacteria; (9) Green non-sulfur bacteria (also anaerobic phototrophs); (10) Radioresistant micrococci and relatives: (11) Thermotoga and Thermosipho thermophiles.

As used herein, the term “fungus” or “fungi” refers in general to any organism from Kingdom Fungi. Historical taxonomic classification of fungi has been according to morphological presentation. Beginning in the mid-1800's, it was recognized that some fungi have a pleomorphic life cycle, and that different nomenclature designations were being used for different forms of the same fungus. In 1981, the Sydney Congress of the International Mycological Association laid out rules for the naming of fungi according to their status as anamorph, teleomorph, or holomorph (Taylor, 2011). With the development of genomic sequencing, it became evident that taxonomic classification based on molecular phylogenetics did not align with morphological-based nomenclature (Shenoy, 2007). As a result, in 2011 the International Botanical Congress adopted a resolution approving the International Code of Nomenclature for Algae, Fungi, and Plants (Melbourne Code) (2012), with the stated outcome of designating “One Fungus=One Name” (Hawksworth, 2012). However, systematics experts have not aligned on common nomenclature for all fungi, nor are all existing databases and information resources inclusive of updated taxonomies. As such, many fungi referenced herein may be described by their anamorph form, but it is understood that based on identical genomic sequencing, any pleomorphic state of that fungus may be considered to be the same organism. For example, the genus Alternaria is the anamorph form of the teleomorph genus Lewia (Kwasna 2003), ergo both would be understood to be the same organism with the same DNA sequence. For example, it is understood that the genus Acremonium is also reported in the literature as genus Sarocladium as well as genus Tilachilidium (Summerbell, 2011). For example, the genus Cladosporium is an anamorph of the teleomorph genus Davidiella (Bensch, 2012), and is understood to describe the same organism. In some cases, fungal genera have been reassigned due to various reasons, and it is understood that such nomenclature reassignments are within the scope of any claimed genus. For example, certain species of the genus Mierodiplodia have been described in the literature as belonging to genus Paraconiothyrium (Crous and Groenveld, 2006).

As used herein, “selectable marker” is a nucleic acid segment that allows one to select for a molecule (e.g., a replicon) or a cell that contains it, often under particular conditions. These markers can encode an activity, such as, but not limited to, production of RNA, peptide, or protein, or can provide a binding site for RNA, peptides, proteins, inorganic and organic compounds or compositions and the like. Examples of selectable markers include but are not limited to: (1) nucleic acid segments that encode products which provide resistance against otherwise toxic compounds (e.g., antibiotics); (2) nucleic acid segments that encode products which are otherwise lacking in the recipient cell (e.g., tRNA genes, auxotrophic markers); (3) nucleic acid segments that encode products which suppress the activity of a gene product; (4) nucleic acid segments that encode products which can be readily identified (e.g., phenotypic markers such as β-galactosidase, green fluorescent protein (GFP), yellow fluorescent protein (YFP), cyan fluorescent protein (CFP), and cell surface proteins); (5) nucleic acid segments that encode products that bind other products which are otherwise detrimental to cell survival and/or function; (6) nucleic acid segments that encode nucleic acids that otherwise inhibit the activity of any of the nucleic acid segments resulting in a visible or selectable phenotype (e.g., antisense oligonucleotides); (7) nucleic acid segments that encode products that bind other products that modify a substrate (e.g. restriction endonucleases); (8) nucleic acid segments that can be used to isolate or identify a desired molecule (e.g. specific protein binding sites); (9) nucleic acid segments that encode a specific nucleotide sequence which can be otherwise non-functional (e.g., for PCR amplification of subpopulations of molecules); and (10) nucleic acid segments, which when absent, directly or indirectly confer resistance or sensitivity to particular compounds.

As used herein, “counterselectable marker” or a “counterselection marker” is a nucleic acid segment that eliminates or inhibits growth of a host organism upon selection. In some embodiments, the counterselectable markers of the present disclosure render the cells sensitive to one or more chemicals/growth conditions/genetic backgrounds. In some embodiments, the counterselectable markers of the present disclosure are toxic genes. In some embodiments, the counterselectable markers are expressed by inducible promoters.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “promoter” refers to a DNA sequence capable of controlling the expression of a coding sequence or functional RNA. The promoter sequence may consist of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter.

The term “competent cell” refers to a cell which has the ability to take up and replicate an exogenous nucleic acid.

As used herein, an “extra-chromosomally replicating plasmid” is an autonomously replicating vector that exists as an extra-chromosomal entity. The replication of an extra-chromosomally replicating plasmid is independent of chromosomal replication.

The term “ribonucleoprotein” as used herein refers to a RNA sequence associated with a protein. The association of RNA and protein may be affected by any suitable means, including, for example, protein-nucleic acid interactions. In other words, the term “ribonucleoprotein” as used herein may refer to a RNA-protein complex.

The term “endonuclease” or “nuclease” refers to any wild-type or mutant enzyme that has the ability to catalyze the hydrolysis (cleavage) of bonds between nucleic acids within a DNA or RNA molecule.

The term “recombinase” generally refers to an enzyme that catalyzes recombination.

The term “transform” refers to the introduction of a molecule, such as a polynucleotide, into a competent cell.

The term “fragment” refers to a portion of a nucleic acid (e.g. a promoter, a gene, an exon, or an intron) or a protein, for example, the portion may comprise about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of a nucleic acid or a protein.

The term “percent identity” in the context of two or more nucleic acid or polypeptide sequences, refers to two or more sequences or subsequences that have a specified percentage of nucleotides or amino acid residues that are the same, when compared. Percentage identity can be calculated using the tools CLUSTALW2 or Basic Local Alignment Search Tool (BLAST), which are available online. The following default parameters may be used for CLUSTALW2 Pairwise alignment: Protein Weight Matrix=Gonnet; Gap Open=10; Gap Extension=0.1.

The term “gene edit” refers to the introduction of a genetic element of interest (e.g., a nucleic acid sequence, a gene of interest, a gene variant, a genetic edit, a single nucleotide polymorphism, a genetic regulatory sequence, a promoter, a non-coding nucleic acid sequence, a terminator, or a combination thereof) at a genomic target site. In embodiments, a gene edit comprises an insertion of a genetic element of interest into the genome of a competent cell, substitution of a genomic target of a competent cell with a genetic element of interest, or generation of a single-nucleotide polymorphism within a competent cell.

The term “gene-editing complex” refers to an enzyme and/or nucleic acid that cleaves a genomic target. Non-limiting examples of gene-editing complexes include ribonucleoproteins (RNPs, e.g., a Cas9 nuclease and a guide RNA), a zinc-finger nuclease (ZFN), and a transcription activator-like effector nuclease (TALEN).

### II. Compositions for Gene Editing

Provided herein are compositions for gene editing comprising:

(a) an extra-chromosomally replicating plasmid comprising a selectable marker gene;

(b) competent cells; and

(c) a gene-editing complex that recognizes a genomic target of a competent cell.

The compositions provided herein are used to introduce one or more gene edits, for example, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 edits, in the genome of a competent cell.

In some embodiments, the compositions provided herein may be used to silence a gene. In some embodiments, the compositions provided herein may be used to upregulate a gene. In some embodiments, the compositions provided herein may be used to mutate a gene.

In some embodiments, the compositions provided herein may be used to introduce a genetic element of interest into a competent cell. In some embodiments, the compositions provided herein are used to remove a genomic target from a competent cell's genome. In some embodiments, the compositions provided herein are used to modify a genomic target within a competent cell's genome. In some embodiments, the compositions provided herein are used to replace a genomic target from a competent cell with a genetic element of interest. In some embodiments, the compositions described herein are used in the methods of the disclosure described in Sections III and IV of this disclosure.

### A. Extra-Chromosomally Replicating Plasmid

In some embodiments, the compositions described herein comprise an extra-chromosomally replicating plasmid. An extra-chromosomally replicating plasmid can maintain replication of a plasmid independently of chromosomal replication. Plasmid replicators and transformation enhancers are DNA fragments optionally found on extra-chromosomally replicating plasmids which enable extrachromosomal maintenance of plasmids.

In some embodiments, the extra-chromosomally replicating plasmids described herein comprise one or more of a plasmid replicator, an autonomously replicating sequence (ARS), and a transformation enhancer.

In some embodiments, the extra-chromosomally replicating plasmid comprises a plasmid replicator. In some embodiments, the plasmid replicator is AMA1. AMA1 is described in detail in Aleksenko et al. Fungal Genetics and Biology 21, 373-387 (1997), which is incorporated by reference herein in its entirety. In some embodiments, the extra-chromosomally replicating plasmid comprises one of the two repeats of AMA1, as described by Fierro et al, and Sarkari et al., each of which is incorporated by reference herein in its entirety: Curr Genet. 1996 April; 29(5):482-9; Sarkari et al. Bioresour Technol. 2017 December; 245(Pt B):1327-1333.

In some embodiments, AMA1 has a nucleic acid sequence of SEQ ID NO: 1 or SEQ ID NO: 2. In some embodiments, AMA1 has a nucleic acid sequence comprising about 95%, about 96%, about 97%, about 98%, about 99%, or about 100% identity to SEQ ID NO: 1 or SEQ ID NO: 2. In some embodiments, AMA1 has a nucleic acid sequence comprising at least about 95%, at least about 96%, at least about 97%, at least about 98%, at least about 99%, or about 100% identity to SEQ ID NO: 1 or SEQ ID NO: 2. In some embodiments, AMA1 has about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 mutations, insertions, or deletions compared to the nucleic acid sequence of SEQ ID NO: 1 or SEQ ID NO: 2. In some embodiments, AMA1 has 1, up to 2, up to 3, up to 4, up to about 5, up to about 6, up to about 7, up to about 8, up to about 9, up to about 10, up to about 11, up to about 12, up to about 13, up to about 14, up to about 15, up to about 16, up to about 17, up to about 18, up to about 19, up to about 20, up to about 21, up to about 22, up to about 23, up to about 24, up to about 25, up to about 26, up to about 27, up to about 28, up to about 29, or up to about 30 mutations, insertions, or deletions compared to the nucleic acid sequence of SEQ ID NO: 1 or SEQ ID NO: 2.

In some embodiments, the extra-chromosomally replicating plasmid comprises a transformation enhancer. In some embodiments, the transformation enhancer is ANS1.

In some embodiments, the extra-chromosomally replicating plasmid comprises a selectable marker gene. In some embodiments, the selectable marker gene is selected from pvrG, hph, nat, amdS, nptII, niaD, and argB. In some embodiments, the selectable marker gene is an antibiotic resistance gene, for example, a chloramphenicol resistance gene, an ampicillin resistance gene, a tetracycline resistance gene, a Zeocin resistance gene, a spectinomycin resistance gene and a Km (Kanamycin resistance gene), tetA (tetracycline resistance gene), G418 (neomycin resistance gene), van (vancomycin resistance gene), tet (tetracycline resistance gene), ampicillin (ampicillin resistance gene), methicillin (methicillin resistance gene), penicillin (penicillin resistance gene), oxacillin (oxacillin resistance gene), erythromycin (erythromycin resistance gene), linezolid (linezolid resistance gene), puromycin (puromycin resistance gene) or a hygromycin (hygromycin resistance gene).

In some embodiments, the extra-chromosomally replicating plasmid comprises a recombinatorial site. In some embodiments, the extra-chromosomally replicating plasmid comprises at least two recombinatorial sites. In some embodiments, the extra-chromosomally replicating plasmid comprises a pair of recombinatorial sites. In some embodiments, the extra-chromosomally replicating plasmid comprises between 1 and 50 recombinatorial sites. In some embodiments, the extra-chromosomally replicating plasmid comprises between 1 and 10 recombinatorial sites. In some embodiments, the extra-chromosomally replicating plasmid may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 recombinatorial sites. Recombinatorial sites are sections or segments of nucleic acid on the participating nucleic acid molecules that are recognized and bound by the recombination proteins during the initial stages of integration or recombination. In some embodiments, the recombinatorial site is a loxP, a Frt, psi, dif, cer, attB, attP, attL, attR, att1, att2, or att site, or mutant, variant, or derivative thereof. In some embodiments, the recombinatorial site is recognized by a recombinase or integrase selected from the group of Cre recombinase. λ-integrase, XerC recombinase, XerD recombinase, flippase (Flp), Flp recombinase, Hin recombinase, Tre recombinase, RecA recombinase, Rad51 recombinase, gamma-delta resolvase, and Dmc1 recombinase. U.S. Pat. Nos. 5,851,808, 7,670,823, and U.S. Publication No. 2014/0296093 describe recombinatorial sites and mutants, variants, or derivatives thereof. Each of these publications is incorporated by reference in its entirety herein.

In some embodiments, the extra-chromosomally replicating plasmid comprises an endonuclease site. In some embodiments, the endonuclease site is at least 10, at least 11, at least 12, at least 13, at least 14, at least 15, at least 16, at least 17, at least 18, at least 19, at least 20, at least 21, at least 22, at least 23, at least 24, or at least 25 nucleotides in length. In some embodiments, the endonuclease site is a homing endonuclease site. A homing endonuclease site comprises a recognition sequence of greater than 10 nucleotides in length. In some embodiments, the extra-chromosomally replicating plasmid comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 endonuclease sites. Non-limiting examples of endonuclease sites include AatII, AbaSI, Acc65I, AccI, AciI, AclI, AcuI, AfeI, AflII, AflIII, AgeI, AhdI, AleI-v2, AluI, AlwI, AlwNI, ApaI, ApaLI, ApeKI, ApoI, AscI, AseI, AsiSI, AvaI, AvalI, AvrII, BaeGI, BaeI, BamHI, BanI, BanII, BbsI, BbvCI, BbvI, BccI, BceAI, BcgI, BciVI, BclI, BcoDI, BfaT, BfuAI, BglI, BglII, BIpI, BmgBI, BmrI, BmtI, BpmI, Bpu10I, BpuEI, BsaAI, BsaBI, BsaHI, BsaI, BsaJI, BsaWI, BsaXI, BseRI, BseYI, BsgI, BsiEI, BsiHKAI, BsiWI, BslI, BsmAI, BsmBI, BsmBI-v2, BsmFI, BsmI, BsoBI, Bsp1286I, BspCNI, BspDI, BspEI, BspHI, BspMI, BspQI, BsrBI, BsrDI, BsrFaI, BsrGI, BsrI, BssHII, BssSaI, BstAPI, BstBI, BstEII, BstNI, BstUI, BstXI, BstYI, Bsu36I, BtgI, BtgZI, BtsCI, BtsIMutI, BtsαI, Cac8L, ClaI, CspCI, CviAII, CviKI-1, CviQI, DdeI, DpnI, DpnII, DraI DraIII, DrdI, EaeI, EagI, EarI, EciI, Eco53kI, EcoNI, EcoOl09I, EcoRI, EcoRV, Esp3I, FatI, FauI, Fnu4HI, FokI, FseI, FspEI, FspI, HaelI, HaeIII, HgaI, HhaI, HincII, HindTII, HinfI, HinPII, HpaI, HpaII, HphI, Hpy166II, Hpy188I, Hpy188III, Hpy99I, HpyAV, HpyCH4III, HpyCH4IV, HpyCH4V, I-CeuI, I-SceI, KasI, KpnI, LpnPI, MboI, MbolI, MfeI, MIuCI, MluI, MIyI, MmeI, MnlI, MscI, MseI, MslT, MspAlI, MspI, MspJI, MwoI, NaeI, Narn, Nb.BbvCI, Nb.BsmI, Nb.BsrDI, Nb.BssSI, Nb.BtsI, NciI, NcoI, NdeI, NgoMIV, NheI, NlaIII, NlaIV, NmeAIII, NotI, NruI, NsiI, NspI, Nt.AlwI, Nt.BbvCI, Nt.BsmAI, Nt.BspQI, Nt.BstNBI, Nt.CviPII, PacI, PaeR7I, PciI, PflFI, PflMI, PI-PspI, PI-SceI, PleI, PluTI, PmeI, PmlI, PpuMI, PshAI, PsiI, PsiI-v2. PspGI, PspOMI, PspXI, PstI, PvuI, PvuII, RsaI, RsrII, Sacd, SacII, SalI, SapI, Sau3AI, Sau96I, SbfI, ScaI, ScrFI, SexAI, SfaNI, SfcI, SfiI, SfoI, SgrAI, SmaI, SmiI, SnaBI, SpeI, SpeI-HF®, SphI, SrfI, SspI, StuI, StyD41, StyI, SwaI, TaqαI, TfiI, TseI, Tsp45I, TspMI, TspRI, Tth111I, XbaI, XcmI, XhoI, XmaI, XmnI, and ZraI. In some embodiments, the homing endonuclease site is I-CeuI, I-SceI, PI-PspI, or PI-SceI. In some embodiments, the endonuclease site is recognized by one or more restriction enzymes.

In some embodiments, the extra-chromosomally replicating plasmid comprises a suicide gene. Examples of suicide genes include, but are not limited to, herpes simplex virus thymidine kinase (HSV-TK), the cytoplasmic domain of Fas, a caspase such as caspase-8 or caspase-9, cytosine deaminase, E1A, FHIT, and other known suicide or apoptosis-inducing genes (Straathof et al., 2005, Blood 105:42474254; Cohen et al., 1999, Leuk. Lymphoma 34:473480; Thomis et al., 2001, Blood 97:1249-1257; Tey et al., 2007, Biol. Blood Marrow Transplant 13:913-924; and Di Stasi et al., 2011, N. Engl. J. Med. 365:1673-1683).

In some embodiments, the suicide gene is under control of an inducible promoter. In some embodiments, the inducible promoter is selected from an alcohol-regulated promoter, a tetracycline-regulated promoter, a steroid regulated promoter, a metal-regulated promoter, a pathogenesis regulated promoter, a carbon-regulated promoter, a heat shock promoter, a synthetic-transcription factor-dependent promoter, a xylose-regulated promoter, or a light-regulated promoter.

In some embodiments, the extra-chromosomally replicating plasmid comprises a carbon-regulated promoter. In some embodiments, the carbon-regulated promoter is controlled by xylose, glucose, sucrose, maltose, ethanol, glycerol, methanol, oleic acid, acetate, hexose, lactose, or galactose. Weinhandl et al. describes many carbon-regulated promoters and is incorporated by reference herein in its entirety: Weinhandl et al. Carbon source dependent promoters in yeasts. Microbial Cell Factories. 2014. 13(5).

In some embodiments, the extra-chromosomally replicating plasmid comprises a barcode. A barcode is any sequence of nucleic acids. In some embodiments, a gene-editing complex or reagent herein recognizes the barcode on the extra-chromosomally replicating plasmid.

### B. Competent Cells

In some embodiments, the compositions and methods of the disclosure use competent cells. Competent cells are cells that take up nucleic acids like DNA. The competent cells utilized in the compositions and methods of the disclosure may be prokaryotic or eukaryotic cells. In some embodiments, the prokaryotic cells are bacteria, for example, species of Escherichia, Klebsiella, Salmonella, Bacillus, Streptomyces, Streptococcus, Shigella, Staphylococcus, Corynebacterium, and Pseudomonas. In some embodiments, the eukaryotic cells are animal cells, for example, human cells or insect cells. In some embodiments, the eukaryotic cells are fungi or yeast. In some embodiments, the eukaryotic cells are filamentous fungal cells. In some embodiments, the filamentous fungal cells are protoplasts.

In some embodiments, the competent cells are provided in a concentration between about 1×10⁵ cells/mL and about 1×10¹⁰ cells/mL, for example, about 1×10⁵ cells/mL, 2×10⁵ cells/mL, 3×10⁵ cells/mL, 4×10⁵ cells/mL, 5×10⁵ cells/mL, 6×10⁵ cells/mL, 7×10⁵ cells/mL, 8×10⁵ cells/mL, 9×10⁵ cells/mL, 1×10⁶ cells/mL, 2×10⁶ cells/mL, 3×10⁶ cells/mL, 4×10⁶ cells/mL, 5×10⁶ cells/mL, 6×10⁶ cells/mL, 7×10⁶ cells/mL, 8×10⁶ cells/mL, 9×10⁶ cells/mL, 1×10⁷ cells/mL, 2×10⁷ cells/mL, 3×10⁷ cells/mL, 4×10⁷ cells/mL, 5×10⁷ cells/mL, 6×10⁷ cells/mL, 7×10⁷ cells/mL, 8×10⁷ cells/mL, 9×10⁷ cells/mL, 1×10⁸ cells/mL, 2×10⁸ cells/mL, 3×10⁸ cells/mL, 4×10⁸ cells/mL, 5×10⁸ cells/mL, 6×10⁸ cells/mL, 7×10⁸ cells/mL, 8×10⁸ cells/mL, 9×10⁸ cells/mL, 1×10⁹ cells/mL, 2×10⁹ cells/mL, 3×10⁹ cells/mL, 4×10⁹ cells/mL, 5×10⁹ cells/mL, 6×10⁹ cells/mL, 7×10⁹ cells/mL, 8×10⁹ cells/mL, 9×10⁹ cells/mL, or 1×10¹⁰ cells/mL.

### Filamentous Fungal Cells

In some embodiments, the competent cell is a filamentous fungal cell. Filamentous fungi form filamentous structures. In some embodiments, the filamentous fungal cell is used to prepare a protoplast. The filamentous fungus cell can be from any filamentous fungus strain known in the art or described herein including holomorphs, teleomorphs or anamorphs thereof. Non-limiting examples of fungus strains include species of Achlya, Acremonium, Aspergillus. Aureobasidium, Bjerkandera, Ceriporiopsis, Cephalosporium, Chrysosporium, Cochiobolus, Coriolus, Corynascus, Cryphonectria, Cryptococcus, Coprinus, Coriolus, Diplodia, Endothis, Filibasidium, Flumicola, Fusarium, Gibberella, Gliocladium, Humicola, Hypocrea, Magnaporthe, Myceliophthora (e.g., Myceliophthora thermophila), Mucor, Neocallimastix, Neurospora, Paecilomyces, Phanerochaete, Penicillium, Pleurotus, Podospora, Phlebia, Piromyces, Pyricularia, Rhizomucor, Rhizopus, Schizophyllum, Scytalidium, Sporotrichum, Talaromyces, Fhermoascus, Fhielavia, Thermoascus, Thielavia. Trametes, Tolypocladium, Trichoderma, Verticilliwn, Volvariella, or teleomorphs, anamorphs, synonyms, or taxonomic equivalents thereof. In some embodiments, the filamentous fungus is Penicillum rubens.

In some embodiments, mutants of the fungal species described herein are used in the compositions and methods of the disclosure. Examples of such mutants are strains that protoplast well: strains that produce primarily protoplasts with a single nucleus: strains that regenerate efficiently in microtiter plates, strains that regenerate faster and/or strains that take up polynucleotide (e.g., DNA) molecules efficiently, strains that have lost the ability to sporulate, slow-growing strains, and strains that produce cultures of low viscosity such as, for example, cells that produce hyphae in culture that are not so entangled as to prevent isolation of single clones and/or raise the viscosity of the culture, strains that have reduced random integration (e.g., disabled non-homologous end joining pathway) or combinations thereof.

In some embodiments, a mutant filamentous fungal strain lacks a selectable marker gene. In some embodiments, the mutant filamentous fungus strain is a uridine-requiring mutant strain. In some embodiments, the mutant strain is deficient in orotidine-5′-phosphate decarboxylase (OMPD), which is encoded by pyrG, or orotate p-ribosyl transferase (OPRT), which is encoded by pyrE. The following articles describe filamentous fungal strains and are incorporated by reference herein in their entirety: T. Goosen et al, Curr Genet. 1987, 11:499 503, J. Begueret et al., Gene. 1984 32:487 92.

In some embodiments, a mutant filamentous fungal strain possesses a compact cellular morphology characterized by shorter hyphae and a more yeast-like appearance. Examples of such mutants are filamentous fungal cells with altered gasl expression as described in U.S. Publication No. 2014/0220689, which is incorporated by reference herein in its entirety.

In some embodiments, a mutant filamentous fungal strain has an altered DNA repair system. In some embodiments, the altered DNA repair system is extremely efficient in homologous recombination and/or extremely inefficient in random integration. The efficiency of targeted integration of a genetic element of interest into the genome of the competent cell by homologous recombination, i.e. integration in a predetermined target locus, can be increased by augmented homologous recombination abilities and/or diminished non-homologous recombination abilities of the host cell. Augmentation of homologous recombination can be achieved by overexpressing one or more genes involved in homologous recombination (e.g., Rad51 and/or Rad52 protein). Removal, disruption or reduction in the activity of one or more non-homologous recombination pathways (e.g., the canonical non-homologous end joining (NHEJ) pathway, the Alternative NHEJ or microhomology-mediated end-joining (Ait-NHEJ/MMEJ) pathway and/or the polymerase theta mediated end-joining (TMEJ) pathway) in the competent cells of the present disclosure can be achieved by any method known in that art such as, for example, by use of an antibody, a chemical inhibitor, a protein inhibitor, a physical inhibitor, a peptide inhibitor, or an anti-sense or RNAi molecule directed against a component of a specific non-homologous recombination (NHR) pathway (e.g., the NHEJ pathway, the Alt-NHEJ/MMEJ pathway and/or the TMEJ pathway).

In some embodiments, the activity of a single non-homologous end joining pathway is inhibited or reduced. In some embodiments, the activity of a combination of non-homologous end-joining pathways is inhibited or reduced such that the activity of one of the non-homologous end-joining pathways remains intact. In some embodiments, the activity of every non-homologous end-joining pathway is reduced or inhibited.

Examples of components of the NHEJ pathway that can be targeted for inhibition or reduction of activity alone or in combination can include, but are not limited to yeast KU70 or yeast KU80 or homologues or orthologs thereof. Examples of components of the Alt-NHEJ/MMEJ pathway that can be targeted for inhibition or a reduction in activity alone or in combination can include, but are not limited to a Polq gene, a Mre11 gene, an XPF-ERCCI gene or homologues or orthologs thereof. An example of a component of the NHEJ/MMEJ pathway that can be targeted for inhibition or a reduction in activity can include, but is not limited to a Polq gene or a homologue or ortholog thereof. In some embodiments, the competent cell is deficient in one or more genes (e.g., yeast KU70, KU80 or homologues or orthologs thereof) of the NHEJ pathway. Examples of such mutants are cells with a deficient hdfA or hdfB gene as described in WO 05/95624, which is incorporated by reference herein in its entirety. In some embodiments, a host-cell for use in the methods provided herein can be deficient in one or more genes of the Alternative NHEJ or microhomology-mediated end-joining (Alt-NHEJ/MMEJ) pathway and/or TMEJ pathway. Examples of such mutants are cells that lack Polq gene or possess a mutant Polq gene as described in Wyatt et al. Essential roles for Polymerase θ mediated end-joining in repair of chromosome breaks Mol Cell. 2016 August 18; 63(4): 662-673.

In some embodiments, the methods and compositions described herein use fungal elements derived from filamentous fungi that may be readily separated from other such elements in a culture medium and are capable of reproducing. In some embodiments, the methods and compositions described herein use a fungal element selected from a spore, propagule, hyphal fragment, protoplast or micropellet.

### Production of Protoplasts

In some embodiments, the filamentous fungi cell is a protoplast. A protoplast is a fungal cell without a cell wall. In some embodiments, protoplasts are generated from filamentous fungi cells using the methods described herein or any known method in the art. Suitable procedures for preparation of protoplasts are known in the art including, for example, those described in EP 238,023 and Yelton et al. (1984, Proc. Natl. Acad. Sci. USA 81:1470-1474), which are incorporated by reference herein in their entirety.

In some embodiments, protoplasts are generated by treating a culture of filamentous fungal cells with one or more lytic enzymes or a mixture thereof. The lytic enzymes can be a beta-glucanase and/or a polygalacturonase.

Following enzymatic treatment, the protoplasts can be isolated using methods known in the art. For example, undigested hyphal fragments can be removed by filtering the mixture through a porous barrier (such as Miracloth) in which the pores range in size from about 1 μm to about 200 μm, for example about 1 μm, about 2 μm, about 3 μm, about 4 μm, about 5 μm, about 6 μm, about 7 μm, about 8 μm, about 9 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 105 μm, about 110 μm, about 115 μm, about 120 μm, about 125 μm, about 130 μm, about 135 μm, about 140 μm, about 145 μm, about 150 μm, about 155 μm, about 160 μm, about 165 μm, about 170 μm, about 175 μm, about 180 μm, about 185 μm, about 190 μm, about 195 μm, or about 200 μm in size.

In some embodiments, a filtrate containing protoplasts is centrifuged to cause the protoplasts to pellet to the bottom of the centrifuge tube. In some embodiments, a buffer of substantially lower osmotic strength is gently applied to the surface of the filtered protoplasts. The layered preparation can be centrifuged, which can cause the protoplasts to accumulate at a layer in the tube in which they are neutrally buoyant. Protoplasts can then be isolated from this layer for further processing. Following protoplast isolation, the remaining enzyme containing buffer can be removed by resuspending the protoplasts in an osmotic buffer and recollected by centrifugation. In some embodiments, the osmotic buffer is 1 M sorbitol buffered using tris(hydroxymethyl)aminomethane (TRIS). After sufficient removal of the enzyme containing buffer, the protoplasts can be resuspended in osmotically stabilized buffer also containing Calcium chloride. In some embodiments, protoplasts are resuspended to a final concentration between about 1×10⁵ protoplasts to about 1×10¹⁰ protoplasts per milliliter (mL). The pre-cultivation and the actual protoplasting step can be varied to optimize the number of protoplasts and the transformation efficiency. Any of the aforementioned steps may be repeated 1 time, 2 times, 3 times, 4 times, 5 times, 6 times, 7 times, 8 times, 9 times, 10 times, or more. Any of the aforementioned parameters may be varied. For example, there can be variations of inoculum size, inoculum method, pre-cultivation media, pre-cultivation times, pre-cultivation temperatures, mixing conditions, washing buffer composition, dilution ratios, buffer composition during lytic enzyme treatment, the type and/or concentration of lytic enzyme used, the time of incubation with lytic enzyme, the protoplast washing procedures and/or buffers, the concentration of protoplasts and/or polynucleotide and/or transformation reagents during the actual transformation, the physical parameters during the transformation, the procedures following the transformation up to the obtained transformants. Protoplasts can be resuspended in an osmotic stabilizing buffer. The composition of such buffers can vary depending on the species, application and needs. In some embodiments, the osmotic stabilizing buffer contains an organic component. Non-limiting examples of organic components include sucrose, citrate, mannitol, or sorbitol. In some embodiments, the osmotic stabilizing buffer contains an inorganic osmotic stabilizing component. Non-limiting examples of inorganic osmotic stabilizing components include KCl, buffers contain an inorganic osmotic stabilizing component like KCl, (NH₄)₂SO₄, MgSO₄, NaCl, or MgCl. Organic or inorganic components may be present in the osmotic stabilizing buffer between about 0.01 M and about 10 M, for example, about 0.01 M, about 0.02 M, about 0.03 M, about 0.04 M, about 0.05 M, about 0.06 M, about 0.07 M, about 0.08 M, about 0.09 M, about 0.1 M, about 0.2 M, about 0.3 M, about 0.4 M, about 0.5 M, about 0.6 M, about 0.7 M, about 0.8 M, about 0.9 M, about 1 M, about 1.1 M, about 1.2 M, about 1.3 M, about 1.4 M, about 1.5 M, about 1.6 M, about 1.7 M, about 1.8 M, about 1.9 M, about 2 M, about 2.1 M, about 2.2 M, about 2.3 M, about 2.4 M, about 2.5 M, about 2.6 M, about 2.7 M, about 2.8 M, about 2.9 M, or about 3 M.

In some embodiments, the osmotic stabilizing buffer is STC (sorbitol, calcium chloride, and TRIS; pH 8.0) or KCl-Citrate (KCl and citrate). In some embodiments, the protoplasts are used in a concentration between about 1×10⁵ cells/mL and about 1×10¹⁰ cells/mL, for example about 1×10⁵ cells/mL, 2×10⁵ cells/mL, 3×10⁵ cells/mL, 4×10⁵ cells/mL, 5×10⁵ cells/mL, 6×10⁵ cells/mL, 7×10⁵ cells/mL, 8×10⁵ cells/mL, 9×10⁵ cells/mL, 1×10⁶ cells/mL, 2×10⁶ cells/mL, 3×10⁶ cells/mL, 4×10⁶ cells/mL, 5×10⁶ cells/mL, 6×10⁶ cells/mL, 7×10⁶ cells/mL, 8×10⁶ cells/mL, 9×10⁶ cells/mL, 1×10⁷ cells/mL, 2×10⁷ cells/mL, 3×10⁷ cells/mL, 4×10⁷ cells/mL, 5×10⁷ cells/mL, 6×10⁷ cells/mL, 7×10⁷ cells/mL, 8×10⁷ cells/mL, 9×10⁷ cells/mL, 1×10⁸ cells/mL, 2×10⁸ cells/mL, 3×10⁸ cells/mL, 4×10⁸ cells/mL, 5×10⁸ cells/mL, 6×10⁸ cells/mL, 7×10⁸ cells/mL, 8×10⁸ cells/mL, 9×10⁸ cells/mL, 1×10⁹ cells/mL, 2×10⁹ cells/mL, 3×10⁹ cells/mL, 4×10⁹ cells/mL, 5×10⁹ cells/mL, 6×10⁹ cells/mL, 7×10⁹ cells/mL, 8×10⁹ cells/mL, 9×10⁹ cells/mL, or 1×10¹⁰ cells/mL. In some embodiments, the protoplasts are used in a concentration between about 1×10⁶ and about 1×10⁹ cells/mL. In some embodiments, the protoplasts are used in a concentration between about 1×10⁷ and about 5×10⁸ cells/mL In some embodiments, the protoplasts are used in a concentration of 1×10⁸ cells/mL.

In some embodiments, after isolation of protoplasts, the protoplasts are cryopreserved. In some embodiments, the protoplasts are mixed with one or more cryoprotectants. The cryoprotectants can be glycols, dimethyl sulfoxide (DMSO), polyols, sugars, 2-Methyl-2,4-pentanediol (MPD), polyvinylpyrrolidone (PVP), methylcellulose, C-linked antifreeze glycoproteins (C-AFGP) or combinations thereof. Glycols for use as cryoprotectants in the methods and systems provided herein can be selected from ethylene glycol, propylene glycol, polypropylene glycol (PEG), glycerol, or combinations thereof. Polyols for use as cryoprotectants in the methods and systems provided herein can be selected from propane-1,2-diol, propane-1,3-diol, 1,1,1-tris-(hydroxymethyl)ethane (THME), and 2-ethyl-2-(hydroxymethyl)-propane-1,3-diol (EHMP), or combinations thereof. Sugars for use as cryoprotectants in the methods and systems provided herein can be selected from trehalose, sucrose, glucose, raffinose, dextrose or combinations thereof. In some embodiments, the protoplasts are mixed with DMSO. DMSO can be mixed with the protoplasts at a final concentration of at least, at most, less than, greater than, equal to, or about 1%, 2%, 3%, 4%, 5%, 6%, 7%, 8%, 9%, 10%, 12.5%, 15%, 20%, 25%, 30%, 35%, 40%, 45%, 50%, 55%, 60%, 65%, 70%, or 75% w/v or v/v. In some embodiments, the cryopreserved protoplasts are distributed to microtiter plates prior to storage. In some embodiments, the cryopreserved protoplasts are stored at a temperature from about −20° C. to about −80° C., for example about −20° C., about −22° C., about −24° C., about −26° C., about −28° C., about −30° C., about −32° C., about −34° C., about −36° C., about −38° C., about −40° C., about −42° C., about −44° C., about −46° C., about −48° C., about −50° C., about −52° C., about −54° C., about −56° C., about −58° C., about −60° C., about −62° C., about −64° C., about −66° C., about −68° C., about −70° C., about −72° C., about −74° C., about −76° C., about −78° C., or about −80° C.

In some embodiments, the protoplasts are stored for about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 1 week, about 2 weeks, about 3 weeks, about 4 weeks, about 5 weeks, about 6 weeks, about 1 month, about 2 months, about 3 months, about 4 months, about 5 months, about 6 months, about 7 months, about 8 months, about 9 months, about 10 months, about 11 months, about 1 year, about 18 months, about 2 years, about 3 years, about 4 years, about 5 years, about 6 years, about 7 years, about 8 years, about 9 years, about 10 years, or more.

C. Gene-Editing Complex that Recognizes a Genomic Target of a Competent Cell

In some embodiments, the compositions described herein comprise a gene-editing complex that recognizes a genomic target (used interchangeably herein with “genomic target site”) of a competent cell. As used herein, a “genomic target” refers to a nucleic acid within a competent cell.

In some embodiments, the genomic target is agene. In some embodiments, the genomic target is an exon, or fragment thereof. In some embodiments, the genomic target is an intron, or fragment thereof. In some embodiments, the genomic target is an intergenic region. The intergenic region could be a promoter, terminator, or other. In some embodiments, a gene-editing complex recognizes more than one target, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more genomic targets. In some embodiments, the compositions comprise 1, 2, 3, 4, 5, 6, 7, 8, 9, 10, 1, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more gene-editing complexes that recognize a genomic target. Each gene-editing complex may recognize the same genomic target or a different genomic target or a combination thereof.

In some embodiments, the length of the genomic target is about 1 bp, about 2 bp, about 3 bp, about 4 bp, about 5 bp, about 6 bp, about 7 bp, about 8 bp, about 9 bp, about 10 bp, about 11 bp, about 12 bp, 13 bp, about 14 bp, about 15 bp, about 16 bp, about 17 bp, about 18 bp, about 19 bp, about 20 bp, about 21 bp, about 22 bp, about 23 bp, about 24 bp, about 25 bp, about 26 bp, about 27 bp, about 28 bp, about 29 bp, about 30 bp, about 31 bp, about 32 bp, about 33 bp, about 34 bp, about 35 bp, about 36 bp, about 37 bp, about 38 bp, about 39 bp, about 40 bp, about 41 bp, about 42 bp, about 43 bp, about 44 bp, about 45 bp, about 46 bp, about 47 bp, about 48 bp, about 49 bp, about 50 bp, about 51 bp, about 52 bp, about 53 bp, about 54 bp, about 55 bp, about 56 bp, about 57 bp, about 58 bp, about 59 bp, about 60 bp, about 61 bp, about 62 bp, about 63 bp, about 64 bp, about 65 bp, about 66 bp, about 67 bp, about 68 bp, about 69 bp, about 70 bp, about 71 bp, about 72 bp, about 73 bp, about 74 bp, about 75 bp, about 76 bp, about 77 bp, about 78 bp, about 79 bp, about 80 bp, about 81 bp, about 82 bp, about 83 bp, about 84 bp, about 85 bp, about 86 bp, about 87 bp, about 88 bp, about 89 bp, about 90 bp, about 91 bp, about 92 bp, about 93 bp, about 94 bp, about 95 bp, about 96 bp, about 97 bp, about 98 bp, about 99 bp, or about 100 bp, about 125 bp, about 150 bp, about 200 bp, about 250 bp, about 300 bp, about 350 bp, about 400 bp, about 450 bp, about 500 bp, about 550 bp, about 600 bp, about 650 bp, about 70) bp, about 750 bp, about 800 bp, about 850 bp, about 900 bp, about 950 bp, about 1 kbp, about 2 kbp, about 3 kbp, about 4 kbp, about 5 kbp, about 6 kbp, about 7 kbp, about 8 kbp, about 9 kbp, about 10 kbp, about 11 kbp, about 12 kbp, 13 kbp, about 14 kbp, about 15 kbp, about 16 kbp, about 17 kbp, about 18 kbp, about 19 kbp, about 20 kbp, about 21 kbp, about 22 kbp, about 23 kbp, about 24 kbp, about 25 kbp, about 26 kbp, about 27 kbp, about 28 kbp, about 29 kbp, about 30 kbp, about 31 kbp, about 32 kbp, about 33 kbp, about 34 kbp, about 35 kbp, about 36 kbp, about 37 kbp, about 38 kbp, about 39 kbp, about 40 kbp, about 41 kbp, about 42 kbp, about 43 kbp, about 44 kbp, about 45 kbp, about 46 kbp, about 47 kbp, about 48 kbp, about 49 kbp, about 50 kbp, about 51 kbp, about 52 kbp, about 53 kbp, about 54 kbp, about 55 kbp, about 56 kbp, about 57 kbp, about 58 kbp, about 59 kbp, about 60 kbp, about 61 kbp, about 62 kbp, about 63 kbp, about 64 kbp, about 65 kbp, about 66 kbp, about 67 kbp, about 68 kbp, about 69 kbp, about 70 kbp, about 71 kbp, about 72 kbp, about 73 kbp, about 74 kbp, about 75 kbp, about 76 kbp, about 77 kbp, about 78 kbp, about 79 kbp, about 80 kbp, about 81 kbp, about 82 kbp, about 83 kbp, about 84 kbp, about 85 kbp, about 86 kbp, about 87 kbp, about 88 kbp, about 89 kbp, about 90 kbp, about 91 kbp, about 92 kbp, about 93 kbp, about 94 kbp, about 95 kbp, about 96 kbp, about 97 kbp, about 98 kbp, about 99 kbp, or about 100 kbp in length.

In some embodiments, the gene-editing complex removes a genomic target from a competent cell.

### Ribonucleoproteins (RNPs)

In some embodiments, the gene-editing complex comprises a ribonucleoprotein (RNP). A RNP comprises a guide RNA (gRNA) and a nuclease. A gRNA is a nucleic acid that guides a nuclease to a target nucleic acid sequence (e.g. a location to be cleaved).

In some embodiments, the target nucleic acid sequence is a genomic target of a competent cell.

In some embodiments, the compositions and methods of the disclosure use 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more RNPs.

In some embodiments, the guide RNA is a single-molecule guide RNA (sgRNA). A sgRNA comprises a spacer sequence and a scaffold sequence. A spacer sequence is a short nucleic acid sequence used to target a nuclease (e.g., a Cas9 nuclease) to a specific nucleotide region of interest (e.g., a genomic DNA sequence to be cleaved). In some embodiments, the spacer may be about 17-24 base pairs in length, such as about 20 base pairs in length. In some embodiments, the spacer may be about 15, about 16, about 17, about 18, about 19, about 20, about 21, about 22, about 23, about 24, about 25, about 26, about 27, about 28, about 29, or about 30 base pairs in length. In some embodiments, the spacer may be at least 15, at least 16, at least 17, at least 18, at least 19, at least 20, at least 21, at least 22, at least 23, at least 24, at least 25, at least 26, at least 27, at least 28, at least 29, or at least 30 base pairs in length. In some embodiments, the spacer may be 15, 16, 17, 18, 19, 20, 21, 22, 23, 24, 25, 26, 27, 28, 29, or 30 base pairs in length. In some embodiments, the spacer sequence has between about 40% to about 80% GC content.

In some embodiments, the spacer targets a site that immediately precedes a 5′ protospacer adjacent motif (PAM). The PAM sequence may be selected based on the desired nuclease. For example, the PAM sequence may be any one of the PAM sequences shown in Table 1 below, wherein N refers to any nucleic acid, R refers to A or G, Y refers to C or T. W refers to A or T, and V refers to A or C or G.

TABLE 1
Exemplary Nucleases and PAM sequences

| PAM sequence | | |
|---|---|---|
| (5′ to 3′) | Nuclease | isolated from |
| NGG | SpCas9 | Streptococcus |
| | | pyogenes |
| NGRRT | SaCas9 | Staphylococcus |
| (SEQ ID NO: 3) | | aureus |
| or | | |
| NGRRN | | |
| (SEQ ID NO: 4) | | |
| NNNNGATT | NmeCas9 | Neisseria |
| (SEQ ID NO: 5) | | meningitidis |
| NNNNRYAC | CjCas9 | Campylobacter |
| (SEQ ID NO: 6) | | jejuni |
| NNAGAAW | StCas9 | Streptococcus |
| (SEQ ID NO: 7) | | thermophiles |
| TTTV | LbCpf1 | Lachnospiraceae |
| (SEQ ID NO: 8) | | bacterium |
| TTTV | AsCpf1 | Acidaminococcus sp. |
| (SEQ ID NO: 8) | | |

In some embodiments, a spacer may target a sequence of a mammalian gene, such as a human gene. In some embodiments, a spacer may target a sequence of a eukaryotic gene, such as a fungal gene. In some embodiments, the spacer may target a mutant gene. In some embodiments, the spacer may target a coding sequence. In some embodiments, the spacer may target an exonic sequence. In some embodiments, a spacer may target an intergenic or non-coding region.

The scaffold sequence is the sequence within the sgRNA that is responsible for nuclease (e.g., Cas9) binding. The scaffold sequence does not include the spacer/targeting sequence. In some embodiments, the scaffold may be about 1 to about 10, about 10 to about 20, about 20 to about 30, about 30 to about 40, about 40 to about 50, about 50 to about 60, about 60 to about 70, about 70 to about 80, about 80 to about 90, about 90 to about 100, about 100 to about 110, about 110 to about 120, or about 120 to about 130 nucleotides in length. In some embodiments, the scaffold may be about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, or about 125 nucleotides in length. In some embodiments, the scaffold may be at least 10, at least 20, at least 30, at least 40, at least 50, at least 60, at least 70, at least 80, at least 90, at least 10, at least 110, at least 120, or at least 125 nucleotides in length.

In some embodiments, the gRNA is a dual-molecule guide RNA, e.g, crRNA and tracrRNA. In some embodiments, the gRNA is selected based on the source microorganism of a nuclease to be associated therewith. In some embodiments, the gRNA may further comprise a polyA tail.

In some embodiments, the gRNA is provided as a linear nucleic acid or as part of a plasmid.

In some embodiments, the nuclease is selected from the group consisting of Cas9, Cas12a (Cpf1), Cas12b, Cas12c, Cas12d, Cas12e, Cas12h, Tnp-B like, Cas13a (C2c2), Cas13b, Cas13c, Cpf1, Cas14, and MAD7, or homologs, orthologs, or paralogs thereof.

In some embodiments, the nuclease is Cas9. The Cas9 protein may be an endonuclease derived from Streptococcus sp., for example, Streptococcus pyogenes or Staphylococcus aureus), but is not limited thereto. In some embodiments, the nuclease has at least 90%, at least 95%, at least 96%, at least 97%, at least 98%, or at least 99% sequence identity to a Cas9 derived from S. aureus (SaCas9) or S. pyogenes.

In some embodiments, the nuclease is Cpf1. In some embodiments, the nuclease has at least 90%, at least 95%, at least 96%, at least 97%, at least 98%, or at least 99% sequence identity to a Cpf1. Examples of the Cpf1 protein include those derived from Parcubacteria bacterium, Lachnospiraceae bacterium, Butyrivibrio proleoclasticus, Peregrinibacteria bacterium, Acidaminococcus sp., Porphyromonas macacae, Lachnospiraceae bacterium. Porphyromonas crevioricanis, Prevotella disiens, Moraxella bovoculi, Smithella sp., Leptospira inadai, Lachnospiraceae bacterium, Francisella novicida, Candidatus Methanoplasma termitum, and Eubacterium eligens, but are not limited thereto.

In some embodiments, the nuclease is isolated from microorganisms. In some embodiments, the nuclease is produced through recombination or synthesis.

In some embodiments, the nuclease is a variant nuclease. A variant RNA-guided endonuclease (e.g, Cas9) has an amino acid sequence that differs by at least one amino acid (e.g, has a deletion, insertion, or substitution) when compared to the amino acid sequence of a wild type nuclease (e.g. Cas9). A variant nuclease may be truncated, fused to another protein (such as another nuclease), or catalytically inactivated. In some embodiments, the variant nuclease has at least 80%, at least 85%, at least 90%, at least 95%, at least 98%, at least 99%, or 100% sequence identity to a naturally occurring Cas9, Cas12a (Cpf1), Cas12b. Cas12c. Cas12d, Cas12e, Cas12h, Tnp-B like, Cas13a (C2c2), Cas13b, Cas13c, Cpf1, Cas14, or MAD7.

In some embodiments, the variant nuclease (e.g, Cas9) can cleave the complementary strand of a target nucleic acid but has reduced ability to cleave the non-complementary strand of a double stranded target nucleic acid.

In some embodiments, the variant nuclease (e.g, Cas9) has a mutation (amino acid substitution) that reduces the function of the RuvC domain of Cas9. As a non limiting example, in some embodiments, a variant Cas9 has a D10A mutation (e.g., aspartate to alanine at an amino acid position corresponding to position 10 of Cas9 encoded by the nucleic acid sequence of and can therefore cleave the complementary strand of a double stranded target nucleic acid but has reduced ability to cleave the non-complementary strand of a double stranded target nucleic acid (thus resulting in a single strand break (SSB) instead of a double strand break (DSB) when the variant Cas9 polypeptide cleaves a double stranded target nucleic acid) (see, for example, Jinek et ah, Science. 2012 Aug. 17; 337(6096):816-21).

In some embodiments, the variant nuclease (e.g., Cas9) can cleave the non-complementary strand of a double stranded target nucleic acid but has reduced ability to cleave the complementary strand of the target nucleic acid. For example, the variant nuclease (e.g., Cas9) can have a mutation (amino acid substitution) that reduces the function of the HNH domain of Cas9. As a non limiting example, in some embodiments, the variant Cas9 can have an H840A mutation (e.g., histidine to alanine at an amino acid position corresponding to position 840 of Streptococcus pyogenes and can therefore cleave the non-complementary strand of the target nucleic acid but has reduced ability to cleave the complementary strand of the target nucleic acid (thus resulting in a single stranded break (SSB) instead of a double stranded break (DSB) when the variant Cas9 polypeptide cleaves a double stranded target nucleic acid).

In some embodiments, the nuclease polypeptide (e.g., Cas9) of the present disclosure can include one or more of the mutations described in the literature, including but not limited to the functional mutations described in: Fonfara et al. Nucleic Acids Res. 2014 February; 42(4):2577-90; Nishimasu H. et al. Cell. 2014 Feb. 27; 156(5):935-49; Jinek M. et al. Science. 2012 337:816-21: Jinek M. et al. Science. 2014 Mar. 14; 343(6176); and Chen et al. Nature. 2017 Oct. 19; 550(7676):407-410; see also U.S. Pat. Pub. No. 2014/0068797; and 2016/0168592; see also PCT Pat. Pub. No. WO 2017/155717: WO 2017/147056; WO 2017/066175; WO 2017/040348: WO 2017/035416; WO 2017/015101; WO 2016/186953; and WO 2016/186745; further, see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; 8,999,641; 9,840,713; 9,840,699; and 9,771.600. Each of the foregoing patents and publications are hereby incorporated by reference in its entirety for all purposes.

In some embodiments, the compositions of the disclosure and methods disclosed herein can be used with a wild type nuclease (e.g., Cas9) having double-stranded nuclease activity, nuclease variants (e.g., Cas9 variants) that act as single-stranded nickases, or other mutants with modified nuclease activity. As such, a nuclease (e.g, Cas9) that is suitable for use in the subject invention can be an enzymatically active nuclease (e.g, Cas9 polypeptide), e.g, can make single- or double-stranded breaks in a target nucleic acid, or alternatively can have reduced enzymatic activity compared to a wild-type RNA-guided endonuclease polypeptide (e.g, Cas9 polypeptide).

The nuclease (e.g, Cas9) can be provided to, or in, a cell in a variety of suitable formats. In some embodiments, the nuclease is encoded by a plasmid. In some embodiments, the nuclease is provided as soluble protein. In some embodiments, the nuclease is provided using lentivirus or adeno-associated viruses.

In some embodiments, the nuclease comprises an element typically used for import into cell nuclei by nuclear transport in eukaryotes (e.g., a nuclear localization signal: NLS).

In some embodiments, the compositions and/or methods comprise a plasmid comprising a gRNA and a nuclease. In some cases, the plasmid or linear nucleic acid contains a sequence for negative selection (e.g. mazF, ccdB, gala-1, lacY, thyA, pheS, tetAR, rpsL, sacB, a temperature sensitive replication origin and the like) and/or flanking recombination sequences such as FLPs, loxP sequences, or the like, that can be activated at a later time for removal of the nuclease encoding sequence.

### TALENS

In some embodiments, the gene-editing complex comprises a transcription activator-like effector nuclease (TALEN) that cleaves a genomic target. A “TALEN” refers to a class of artificial restriction endonucleases that comprises a TAL effector DNA binding domain and a DNA cleavage domain. The target nucleic acid comprises a genomic target of the competent cell. A TALEN induces a site-specific double stranded DNA break in a genomic target.

In some embodiments, the TALEN is a monomeric TALEN that can cleave double stranded DNA without assistance from another TALEN. The term “TALEN” is also used to refer to one or both members of a pair of TALENs that are engineered to work together to cleave DNA at the same site. TALENs that work together can be referred to as a left-TALEN and a right-TALEN, which references the handedness of DNA.

In some embodiments, the DNA cleavage domain of the TALEN comprises any nuclease or fragment thereof described throughout this disclosure. In some embodiments, the DNA cleavage domain is derived from a class of non-specific DNA cleavage domains (e.g., the DNA cleavage domain of type II restriction enzymes). In certain embodiments, the DNA cleavage domain is derived from a type II restriction enzyme (FokI).

In some embodiments, the compositions of the disclosure encode an mRNA encoding for a TALEN. In some embodiments, the compositions comprise a plasmid encoding a TALEN. In some embodiments, the compositions comprise a soluble TALEN protein.

### Zinc Finger Nuclease

In some embodiments, the gene-editing complex comprises a zinc-finger nuclease. As used herein, a “zinc-finger nuclease” or “ZFN” refers to a chimeric protein molecule comprising at least one zinc finger DNA binding domain linked to at least one nuclease capable of cleaving DNA. The zinc finger DNA binding domain recognizes a genomic target, and the nuclease cleaves the genomic target.

In some embodiments, the zinc finger DNA binding domain is at the N-terminus of the chimeric protein molecule and the DNA cleavage domain is located at the C-terminus of this molecule. In some embodiments, the zinc finger DNA binding domain is at the C-terminus of the chimeric protein molecule and the DNA cleavage domain is located at the N-terminus of this molecule.

In some embodiments, the DNA binding domain of the ZFN comprises at least one zinc finger DNA binding domain, for example, 1, 2, 3, 4, 5, 6, 7, 8, 9, 10, or more zinc finger DNA binding domains. Each zinc finger DNA binding domain binds a genomic target. Two zinc finger DNA binding domains within a ZFN may recognize the same or different genomic targets. The region of DNA between the two zinc finger DNA binding domains is referred to as a “spacer.” In some embodiments, the spacer comprises between 1 and 300 base pairs of DNA.

The zinc finger domains of the present invention can be derived from any class or type of zinc finger. In certain embodiments, the zinc finger domain comprises a Cys₂ His₂ type zinc finger, typically represented by, for example, the zinc finger transcription factor TFIIIA or Sp1. DNA recognition specificity and/or binding specificity of ZFN may be varied to achieve the targeted genetic recombination at any genomic target. Such modifications could be accomplished using known molecular biological synthetic techniques and/or chemical synthesis techniques. ZFNs comprising zinc fingers with a wide variety of DNA recognition and/or binding specificities are within the scope of the present invention. In some embodiments, the zinc finger domain is a gag knuckle, a treble clef finger, a zinc ribbon, a Zn2/Cys6-like finger, a TAZ2-domain like, a short zinc-binding loop, or a metallothionein. Krishna et al. describes zinc finger domains in detail and is incorporated by reference herein in its entirety: Krishna et al. Nucleic Acids Res. 2003 Jan. 15; 31(2):532-50. Zinc finger binding domains can be “engineered” to bind to a predetermined nucleotide sequence. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database storing information of existing ZFP designs and binding data.

ZFN DNA cleavage domains may comprise any nuclease or fragment thereof described throughout this disclosure. In some embodiments, the ZFN DNA cleavage domain is derived from a class of non-specific DNA cleavage domains (e.g., the DNA cleavage domain of type II restriction enzymes). In certain embodiments, the DNA cleavage domain is derived from a type II restriction enzyme (Fok1).

The linker between the cleavage domain of ZFN and the recognition domain, if present, contains a sequence of selected amino acid residues, so that the resulting linker is flexible. Alternatively, linkerless constructs are made for maximum target site specificity. A linker-free construct has strong preference for binding to recognition sites and then cleaving between recognition sites 6 bp apart. However, with a linker length between 0 and 18 amino acids long, ZFN-mediated cleavage is present between recognition sites 5-35 bp apart. For a given linker length, there are limitations on the distance between recognition sites that are consistent with both binding and dimerization. In some embodiments, there is no linker between the cleavage domain and the recognition domain, and the target position comprises two 9 nucleotide recognition sites separated by a 6 nucleotide spacer in an inverted orientation with respect to each other.

In some embodiments, the compositions of the disclosure encode an mRNA encoding for a ZFN. In some embodiments, the compositions comprise a plasmid encoding a ZFN. In some embodiments, the compositions comprise a soluble ZFN protein.

### D. A Genetic Element of Interest

In some embodiments, the compositions of the disclosure comprise a genetic element of interest. As used herein, a “genetic element of interest” is a nucleic acid that is introduced at a genomic target site. In some embodiments, the genetic element of interest is a deoxyribonucleic acid (DNA). In some embodiments, the genetic element of interest is a ribonucleic acid (RNA). In some embodiments, the compositions do not comprise a genetic element of interest. In some embodiments, the compositions comprise between about 1 and about 100 genetic elements of interest. In some embodiments, the compositions comprise between about 1 and about 10 genetic elements of interest. For example, in some embodiments, the compositions comprise about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100 genetic elements of interest.

In some embodiments, the genetic element of interest comprises a region of homology to the genome of the competent cell (e.g., target genome). In some embodiments, a region of homology to the target genome of the genetic element of interest is found at the 5′ end of the genetic element of interest. In some embodiments, a region of homology to the target genome of the genetic element of interest is found at the 3′ end of the genetic element of interest. In some embodiments, a region of homology to the target genome of the genetic element of interest is found at the 5′ and 3′ end of the genetic element of interest.

In some embodiments, the genetic element of interest is introduced at a genomic target site. In some embodiments, the genetic element of interest replaces a genomic target site.

In some embodiments, the genomic target site is a promoter region. In some embodiments, the genomic target site is a terminator region. In some embodiments, the genomic target site is a coding region. In some embodiments, the genomic target site is a non-coding region.

In some embodiments, the genetic element of interest is selected from the group consisting of: a nucleic acid sequence, a gene of interest, a gene variant, a genetic edit, a single nucleotide polymorphism, a genetic regulatory sequence, a promoter, a non-coding nucleic acid sequence, a terminator, or any combination thereof. In some embodiments, the genetic element of interest is a biosynthetic gene cluster. A biosynthetic gene cluster is an organized group of genes responsible for the production of one or more compounds.

In some embodiments, the genetic element of interest is a nucleic acid sequence. Nucleic acids comprise nucleotides. In some embodiments, nucleotides contain ribose, deoxyribose, or analogs thereof, for example, 2-O-methyl, 2′-O-allyl, 2′-fluoro or 2′-Azidoribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogues such as methyl riboside. In some embodiments, one or more phosphodiester bonds of a nucleic acid may be replaced with alternative groups. Alternative groups include, but are not limited to P(O)S (“thioate”), P(S)S (“dithioate”), (O)NR2 (“amidate”), P(O)R, P(O)OR′, CO or CH2 (“formacetal”), in which each R or R′ is independently H or substituted or unsubstituted alkyl (1-20 C), optionally an ether-(—O—)-bond, aryl, alkenyl, cycloalkyl, cycloalkenyl or araldyl. Not all bonds in a polynucleotide must be identical. The foregoing description is applicable to all of the nucleic acids referred to herein, including RNA and DNA.

In some embodiments, the nucleotide or nucleic acid is labeled. In some embodiments, a nucleotide is labeled according to methods known in the art. In some embodiments, the nucleotide is labeled with a dye and/or a detectable moiety such as a specific binding pair member (e.g. biotin-avidin). Labeled” dNTP or rNTP may also be indirect be marked by its attachment to, for example, a component to which a marker is/may be attached. A dNTP or rNTP may comprise a molecular moiety (for example, an amino group or hydrazide group) to which a label is attached. Non-limiting examples of labels include fluorescent dyes (e.g., fluorescein isothiocyanate, Texas Red, rhodamine, green fluorescent protein and the like), radioisotopes (e.g. ³H, ³⁵S, ³²P, ³³P, ¹²⁵I or ¹⁴C), enzymes (e.g. LacZ, horseradish peroxidase, alkaline phosphatase), digoxigenin, and colorimetric labels such as colloidal gold or colored glass or plastic beads (e.g., polystyrene, polypropylene, latex, etc.). Various anti-ligands and ligands may be used (as labels themselves or as a label attachment agent).

In some embodiments, the nucleic acid sequence is about 10 base pairs (bp), about 20 bp, about 30 bp, about 40 bp, about 50 bp, about 60 bp, about 70 bp, about 80 bp, about 90 bp, about 100 bp, about 150 bp, about 200 bp, about 250 bp, about 300 bp, about 350 bp, about 400 bp, about 450 bp, about 500 bp, about 550 bp, about 600 bp, about 650 bp, about 700 bp, about 750 bp, about 800 bp, about 850 bp, about 900 bp, about 950 bp, about 1 kilobase pair (kbp), at least 2 kbp, at least 3 kbp, at least 4 kbp, at least 5 kbp, at least 6 kbp, at least 7 kbp, at least 8 kbp, at least 9 kbp, at least 10 kbp, at least 11 kbp, at least 12 kbp, about 13 kbp, about 14 kbp, about 15 kbp, about 16 kbp, about 17 kbp, about 18 kbp, about 19 kbp, about 20 kbp, about 21 kbp, about 22 kbp, about 23 kbp, about 24 kbp, about 25 kbp, about 26 kbp, about 27 kbp, about 28 kbp, about 29 kbp, about 30 kbp, about 31 kbp, about 32 kbp, about 33 kbp, about 34 kbp, about 35 kbp, about 36 kbp, about 37 kbp, about 38 kbp, about 39 kbp, about 40 kbp, about 41 kbp, about 42 kbp, about 43 kbp, about 44 kbp, about 45 kbp, about 46 kbp, about 47 kbp, about 48 kbp, about 49 kbp, about 50 kbp, about 51 kbp, about 52 kbp, about 53 kbp, about 54 kbp, about 55 kbp, about 56 kbp, about 57 kbp, about 58 kbp, about 59 kbp, about 60 kbp, about 61 kbp, about 62 kbp, about 63 kbp, about 64 kbp, about 65 kbp, about 66 kbp, about 67 kbp, about 68 kbp, about 69 kbp, about 70 kbp, about 71 kbp, about 72 kbp, about 73 kbp, about 74 kbp, about 75 kbp, about 76 kbp, about 77 kbp, about 78 kbp, about 79 kbp, about 80 kbp, about 81 kbp, about 82 kbp, about 83 kbp, about 84 kbp, about 85 kbp, about 86 kbp, about 87 kbp, about 88 kbp, about 89 kbp, about 90 kbp, about 91 kbp, about 92 kbp, about 93 kbp, about 94 kbp, about 95 kbp, about 96 kbp, about 97 kbp, about 98 kbp, about 99 kbp, or about 100 kbp in length.

In some embodiments, the genetic element of interest is a gene (referred to interchangeably as a “gene of interest.” In some embodiments, the genetic element of interest comprises multiple genes, for example, about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 genes. In some embodiments, the gene is exogenous to the competent cell. In some embodiments, the gene is endogenous to the competent cell. In some embodiments, the gene of interest encodes an enzyme, a substrate-binding protein, a surface-active protein, a transporter, a regulatory protein or a structural protein. In some embodiments, the “gene of interest” can be located intracellularly or extracellularly. In some embodiments, the product of a gene of interest is a secreted protein. In some embodiments, the gene of interest comprises a mutation compared to the wild-type gene of interest. The mutation can be an insertion, deletion, substitution, or single-nucleotide polymorphism. In some embodiments, the gene comprises a genetic regulatory or control element (e.g. a promoter or a terminator). In some embodiments, the gene is flanked by a genetic regulatory or control element (e.g. a promoter or a terminator).

In some embodiments, the genetic element of interest is a promoter or a terminator sequence. The promoter sequence and/or terminator sequence can be endogenous or heterologous relative to the variant strain and/or the parental strain. Promoter sequences can be operably linked to the 5′ termini of the sequences to be expressed. A variety of known fungal promoters are likely to be functional in the disclosed host strains such as, for example, the promoter sequences of C1 endoglucanases, the 55 kDa cellobiohydrolase (CBHl), glyceraldehyde-3-phosphate dehydrogenase A. C. lucknowense GARG 27K and the 30 kDa xylanase (XylF) promoters from Chrysosporium, as well as the Aspergillus promoters described in, e.g. U.S. Pat. Nos. 4,935,349; 5,198,345; 5,252,726; 5,705,358; and 5,965,384; and PCX application WO 93/07277. Terminator sequences can be operably linked to the 3′ termini of the sequences to be expressed. A variety of known fungal terminators are likely to be functional in the disclosed host strains. Examples are the A. nidulans trpC terminator, A. niger alpha-glucosidase terminator, A. niger glucoamylase terminator, Mucor miehei carboxyl protease terminator (see U.S. Pat. No. 5,578,463), Chrysosporium terminator sequences, e.g. the EG6 terminator, and the Trichoderma reesei cellobiohydrolase terminator.

In some embodiments, the genetic element of interest is a gene edit. A gene edit may be an insertion of a genetic element of interest into the genome of a competent cell, substitution of a genomic target of a competent cell with a genetic element of interest, or generation of a single-nucleotide polymorphism within a competent cell.

In some embodiments, the genetic element of interest is a single nucleotide polymorphism.

In some embodiments, the genetic element of interest is a genetic regulatory sequence.

In some embodiments, the genetic element of interest is a non-coding nucleic acid sequence.

In some embodiments, the genetic element of interest is linear, single-stranded DNA. In some embodiments, the genetic element of interest is linear, double-stranded DNA. In some embodiments, the genetic element of interest comprises one or more sticky ends. As used herein, a “sticky end” is a region of unpaired nucleotides at the end of a DNA double helix. In some embodiments, the genetic element of interest is linear.

In some embodiments, the genetic element of interest is a vector. In some embodiments, a vector comprises a genetic element of interest. In some embodiments, the vector is an integrative vector. An integrative vector becomes integrated into the genome and replicated together with the chromosome(s) into which it has been integrated. An integrative vector may integrate at random or at a predetermined genomic target site of a competent cell.

In some embodiments, an integrative vector comprises a DNA fragment, which is homologous to a DNA sequence in a predetermined target locus in the genome of the competent cell for targeting the integration of the vector to this predetermined locus. In some embodiments, an integrative vector is linearized prior to transformation of the cell. Linearization is performed such that at least one but preferably either end of the vector is flanked by sequences homologous to the target locus. In some embodiments, the length of the homologous sequences flanking the target locus is at least 10 base pairs (bp), at least 20 bp, at least 30 bp, at least 40 bp, at least 50 bp, at least 60 bp, at least 70 bp, at least 80 bp, at least 90 bp, at least 100 bp, at least 150 bp, at least 200 bp, at least 250 bp, at least 300 bp, at least 350 bp, at least 400 bp, at least 450 bp, at least 500 bp, at least 550 bp, at least 600 bp, at least 650 bp, at least 700 bp, at least 750 bp, at least 800 bp, at least 850 bp, at least 900 bp, at least 950 bp, at least 1 kilobase pair (kbp), at least 2 kbp, at least 3 kbp, at least 4 kbp, at least 5 kbp, at least 6 kbp, at least 7 kbp, at least 8 kbp, at least 9 kbp, at least 10 kbp, at least 11 kbp, at least 12 kbp, at least 13 kbp, at least 14 kbp, at least 15 kbp, at least 16 kbp, at least 17 kbp, at least 18 kbp, at least 19 kbp, at least 20 kbp, at least 21 kbp, at least 22 kbp, at least 23 kbp, at least 24 kbp, at least 25 kbp, at least 26 kbp, at least 27 kbp, at least 28 kbp, at least 29 kbp, at least 30 kbp, at least 31 kbp, at least 32 kbp, at least 33 kbp, at least 34 kbp, at least 35 kbp, at least 36 kbp, at least 37 kbp, at least 38 kbp, at least 39 kbp, at least 40 kbp, at least 41 kbp, at least 42 kbp, at least 43 kbp, at least 44 kbp, at least 45 kbp, at least 46 kbp, at least 47 kbp, at least 48 kbp, at least 49 kbp, at least 50 kbp, at least 51 kbp, at least 52 kbp, at least 53 kbp, at least 54 kbp, at least 55 kbp, at least 56 kbp, at least 57 kbp, at least 58 kbp, at least 59 kbp, at least 60 kbp, at least 61 kbp, at least 62 kbp, at least 63 kbp, at least 64 kbp, at least 65 kbp, at least 66 kbp, at least 67 kbp, at least 68 kbp, at least 69 kbp, at least 70 kbp, at least 71 kbp, at least 72 kbp, at least 73 kbp, at least 74 kbp, at least 75 kbp, at least 76 kbp, at least 77 kbp, at least 78 kbp, at least 79 kbp, at least 80 kbp, at least 81 kbp, at least 82 kbp, at least 83 kbp, at least 84 kbp, at least 85 kbp, at least 86 kbp, at least 87 kbp, at least 88 kbp, at least 89 kbp, at least 90 kbp, at least 91 kbp, at least 92 kbp, at least 93 kbp, at least 94 kbp, at least 95 kbp, at least % kbp, at least 97 kbp, at least 98 kbp, at least 99 kbp, at least 100 kbp, or more.

### E. Additional Reagents

In some embodiments, the compositions of the disclosure comprise one or more additional reagents. In some embodiments, the additional reagent is utilized to remove an extra-chromosomally replicating plasmid from a competent cell. In some embodiments, the additional reagent maintains the pH of the composition. In some embodiments, the reagent is a recombinase, an integrase, an endonuclease, a RNP, a transcription activator-like effector nuclease (TALEN), a zinc-finger nuclease (ZFN), alcohol, a transcription factor, tetracycline, a steroid, a metal, heat, light, an antibiotic, a sugar, xylose, glucose, sucrose, maltose, ethanol, glycerol, methanol, oleic acid, acetate, hexose, lactose, galactose, a buffer, or a salt.

In some embodiments, the reagent recognizes a recombinatorial site, a restriction endonuclease site, a selectable marker gene, or another nucleotide sequence. In some embodiments, the reagent recognizes a recombinatorial site, restriction endonuclease site, a selectable marker gene, or another nucleotide sequence on an extra-chromosomally replicating plasmid.

In some embodiments, the length of the recombinatorial site, restriction endonuclease site, or selectable marker gene is about 1 bp, about 2 bp, about 3 bp, about 4 bp, about 5 bp, about 6 bp, about 7 bp, about 8 bp, about 9 bp, about 10 bp, about 11 bp, about 12 bp, 13 bp, about 14 bp, about 15 bp, about 16 bp, about 17 bp, about 18 bp, about 19 bp, about 20 bp, about 21 bp, about 22 bp, about 23 bp, about 24 bp, about 25 bp, about 26 bp, about 27 bp, about 28 bp, about 29 bp, about 30 bp, about 31 bp, about 32 bp, about 33 bp, about 34 bp, about 35 bp, about 36 bp, about 37 bp, about 38 bp, about 39 bp, about 40 bp, about 41 bp, about 42 bp, about 43 bp, about 44 bp, about 45 bp, about 46 bp, about 47 bp, about 48 bp, about 49 bp, about 50 bp, about 51 bp, about 52 bp, about 53 bp, about 54 bp, about 55 bp, about 56 bp, about 57 bp, about 58 bp, about 59 bp, about 60 bp, about 61 bp, about 62 bp, about 63 bp, about 64 bp, about 65 bp, about 66 bp, about 67 bp, about 68 bp, about 69 bp, about 70 bp, about 71 bp, about 72 bp, about 73 bp, about 74 bp, about 75 bp, about 76 bp, about 77 bp, about 78 bp, about 79 bp, about 80 bp, about 81 bp, about 82 bp, about 83 bp, about 84 bp, about 85 bp, about 86 bp, about 87 bp, about 88 bp, about 89 bp, about 90 bp, about 91 bp, about 92 bp, about 93 bp, about 94 bp, about 95 bp, about 96 bp, about 97 bp, about 98 bp, about 99 bp, or about 100 bp, about 125 bp, about 150 bp, about 200 bp, about 250 bp, about 300 bp, about 350 bp, about 400 bp, about 450 bp, about 500 bp, about 550 bp, about 600 bp, about 650 bp, about 700 bp, about 750 bp, about 800 bp, about 850 bp, about 90 bp, about 950 bp, about 1 kbp, about 2 kbp, about 3 kbp, about 4 kbp, about 5 kbp, about 6 kbp, about 7 kbp, about 8 kbp, about 9 kbp, about 10 kbp, about 11 kbp, about 12 kbp, 13 kbp, about 14 kbp, about 15 kbp, about 16 kbp, about 17 kbp, about 18 kbp, about 19 kbp, about 20 kbp, about 21 kbp, about 22 kbp, about 23 kbp, about 24 kbp, about 25 kbp, about 26 kbp, about 27 kbp, about 28 kbp, about 29 kbp, about 30 kbp, about 31 kbp, about 32 kbp, about 33 kbp, about 34 kbp, about 35 kbp, about 36 kbp, about 37 kbp, about 38 kbp, about 39 kbp, about 40 kbp, about 41 kbp, about 42 kbp, about 43 kbp, about 44 kbp, about 45 kbp, about 46 kbp, about 47 kbp, about 48 kbp, about 49 kbp, about 50 kbp, about 51 kbp, about 52 kbp, about 53 kbp, about 54 kbp, about 55 kbp, about 56 kbp, about 57 kbp, about 58 kbp, about 59 kbp, about 60 kbp, about 61 kbp, about 62 kbp, about 63 kbp, about 64 kbp, about 65 kbp, about 66 kbp, about 67 kbp, about 68 kbp, about 69 kbp, about 70 kbp, about 71 kbp, about 72 kbp, about 73 kbp, about 74 kbp, about 75 kbp, about 76 kbp, about 77 kbp, about 78 kbp, about 79 kbp, about 80 kbp, about 81 kbp, about 82 kbp, about 83 kbp, about 84 kbp, about 85 kbp, about 86 kbp, about 87 kbp, about 88 kbp, about 89 kbp, about 90 kbp, about 91 kbp, about 92 kbp, about 93 kbp, about 94 kbp, about 95 kbp, about 96 kbp, about 97 kbp, about 98 kbp, about 99 kbp, or about 100 kbp in length.

### Ribonucleoproteins

In some embodiments, the compositions comprise a RNP that recognizes a site on the extra-chromosomally replicating plasmid. In some embodiments, the RNP recognizes the selectable marker gene. In some embodiments, the site is 500 base pairs or less from a terminus of the selectable marker gene on the extra-chromosomally replicating plasmid.

In some embodiments, the compositions and methods of the disclosure use 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more RNPs.

A RNP that recognizes a site on the extra-chromosomally replicating plasmid comprises a gRNA and a nuclease. Characteristics of an RNP that recognizes a site on the extra-chromosomally replicating plasmid are described in Section IIC of this disclosure. In some embodiments, the gRNA is an sgRNA. In some embodiments, the nuclease is Cas9.

Recombinases and/or Integrases

In some embodiments, the compositions comprise a recombinase or an integrase. A recombinase is an enzyme which promotes genetic recombination. In some embodiments, integrases promote genetic recombination by integrating DNA into a cell. In some embodiments, genetic recombination is site-specific, e.g. the recombinase recognizes a specific sequence of DNA. In some embodiments, the recombinase recognizes a recombinatorial site.

Non-limiting examples of recombinases and/or integrases include Cre recombinase, %-integrase, XerC recombinase, XerD recombinase, flippase (Flp), Flp recombinase, Hin recombinase, Tre recombinase, RecA recombinase, Rad51 recombinase, gamma-delta resolvase, and Dmc1 recombinase.

### Endonuclease

In some embodiments, the compositions comprise an endonuclease. In some embodiments, the endonuclease recognizes and cleaves DNA at an endonuclease site. Non-limiting examples of endonucleases include AatII, AbaSI, Acc65I, AccI, AciI, AclI, AcuI, AfeI, AflII, AflIII, AgeI, AhdI, AleI-v2, AluI, AlwI, AlwNI, ApaI, ApaLI, ApeKI, ApoI, AscI, AseI, AsiSI, AvaI, AvaII, AvrII, BaeGI, BaeI, BamHI, BanI, BanII, BbsI, BbvCI, BbvI, BccI, BceAI, BcgI, BciVI, BclI, BcoDI, BfaI, BfuAI, BglI, BglII, BlpI, BmgBI, BmrI, BmtI, BpmI, Bpu10I, BpuEI, BsaAI, BsaBI, BsaHI, BsaI, BsaJI, BsaWI, BsaXI, BseRI, BseYI, BsgI, BsiEI. BsiHKAI, BsiWI, BslI, BsmAI, BsmBI, BsmBI-v2, BsmFI, BsmI, BsoBI, Bsp1286I, BspCNI, BspDI, BspEI, BspHI, BspMI, BspQI, BsrBI, BsrDI, BsrFaI, BsrGI, BsrI, BssHII, BssSaI, BstAPI, BstBI, BstEII, BstNI, BstUI, BstXI, BstYI, Bsu36I, BtgI, BtgZI, BtsCI, BtsIMutI, BtsαI, Cac8I, ClaI, CspCI, CviAII, CviKI-1, CviQI, DdeI, DpnI, DpnII, DraI, DraIII, DrdI, EaeI, EagI, EarI, EciI, Eco53kI, EcoNI, EcoO109I, EcoRI, EcoRV, Esp3I, FatI, FauI, Fnu4HI, FokI, FseI, FspEI, FspI, HaelI, HaeII, HgaI, HhaI, HincII, HindIII, HinfI, HinP1I, HpaI, HpaII, HphI, Hpy166II, Hpy188I, Hpy188III, Hpy99I, HpyAV, HpyCH4III, HpyCH4IV, HpyCH4V, I-CeuI, I-SceI, KasI, KpnI, LpnPI, MboI, MboII, MfeI, MIuCI, MluI, MlyI, MmeI, MnlI, MscI, MseI, MslI, MspAlI, MspI, MspJI, MwoI, NaeI, NarI, Nb.BbvCI, Nb.BsmI, Nb.BsrDI, Nb.BssSI, Nb.BtsI, NciI, NcoI, NdeI, NgoMIV, NheI, NlaIII, NlaIV, NmeAIII, NotI, NruI, NsiI, NspI, Nt.AlwI, Nt.BbvCI, Nt.BsmAI, Nt.BspQI, Nt.BstNBI, Nt.CviPII, PacI, PaeR7I, PciI, PflFI, PflMI, PI-PspI, PI-SceI, PleI, PluTI, PmeI, PmlI, PpuMI, PshAI, PsiI, PsiI-v2, PspGI, PspOMI, PspXI, PstI, PvuI, PvuII, RsaI, RsrII, SacI, SacII, SalI, SapI, Sau3AI, Sau96I, SbfI, ScaI, ScrFI, SexAI, SfaNI, SfcI, SfiI, SfoI, SgrAI, SmaI, SmiI, SnaBI, SpeI, SpeI-HF®, SphI, SrfI, SspI, StuI, StyD4I, StyI, SwaI, TaqαI, TfiI, TseI, Tsp451, TspMI, TspRI, Tth111I, XbaI, XcmI, XhoI, XmaI, XmnI, and ZraI, In some embodiments, the restriction endonuclease is a homing endonuclease. In some embodiments, the homing endonuclease site is I-CeuI, I-SceI, PI-PspI, or PI-SceI.

### Other Reagents

In some embodiments, the composition comprises an alcohol, a transcription factor, tetracycline, a steroid, a metal, heat, or light. In some embodiments, the compositions contain a reagent that regulates expression of a suicide gene. For example, the reagent may induce or enhance expression of a suicide gene. In some embodiments, the reagent is a carbon source. Non-limiting examples of carbon sources include xylose, glucose, sucrose, maltose, ethanol, glycerol, methanol, oleic acid, acetate, hexose, lactose, or galactose.

In some embodiments, the composition comprises agents that regulate pH (e.g. buffers).

In some embodiments, the composition comprises a salt. Non-limiting examples of salts include sodium chloride, potassium chloride, ammonium chloride, sodium acetate, sodium citrate, copper sulfate, sodium iodide, and sodium sulfate.

### III. Methods for Recycling Extra-Chromosomally Replicating Plasmids

Extra-chromosomally replicating plasmids enable gene editing without integration of the selectable marker gene in the genome of the competent cell, so called ‘marker-free’ gene editing. However, the difficulties associated with removing extra-chromosomally replicating plasmids, limits their usefulness for making multiple genetic edits in a competent cell. Described herein are superior strategies for recycling extra-chromosomally replicating plasmids.

In some embodiments, the method for removing an extra-chromosomally replicating plasmid from a competent cell comprises administering a reagent to remove the extra-chromosomally replicating plasmid.

In some embodiments, the methods comprise removing an extra-chromosomally replicating plasmid from competent cells transformed with the compositions of the disclosure (e.g. those described in Section II of this disclosure). The method utilizes competent cells and extra-chromosomally replicating plasmids as described in Section II. For example, the competent cell may be a eukaryotic cell, a prokaryotic cell, a fungal cell, or a filamentous fungal cell (e.g., a protoplast), and the extra-chromosomally replicating plasmid may comprise any combination of a selectable marker gene, a recombinatorial site, an endonuclease site, and a suicide gene. In some embodiments, the extra-chromosomally replicating plasmid comprises a plasmid replicator. In some embodiments, the plasmid replicator is AMA1. In some embodiments, the competent cells are transformed according to the methods in Section IV of this disclosure. Transformation is evaluated by selecting for cells that comprise the extra-chromosomally replicating plasmid as described in Section IV of this disclosure.

In some embodiments, the method of recycling an extra-chromosomally replicating plasmid comprises administering a reagent to remove the extra-chromosomally replicating plasmid.

### Recycling of Extra-Chromosomally Replicating Plasmid Through Introduction of an RNP

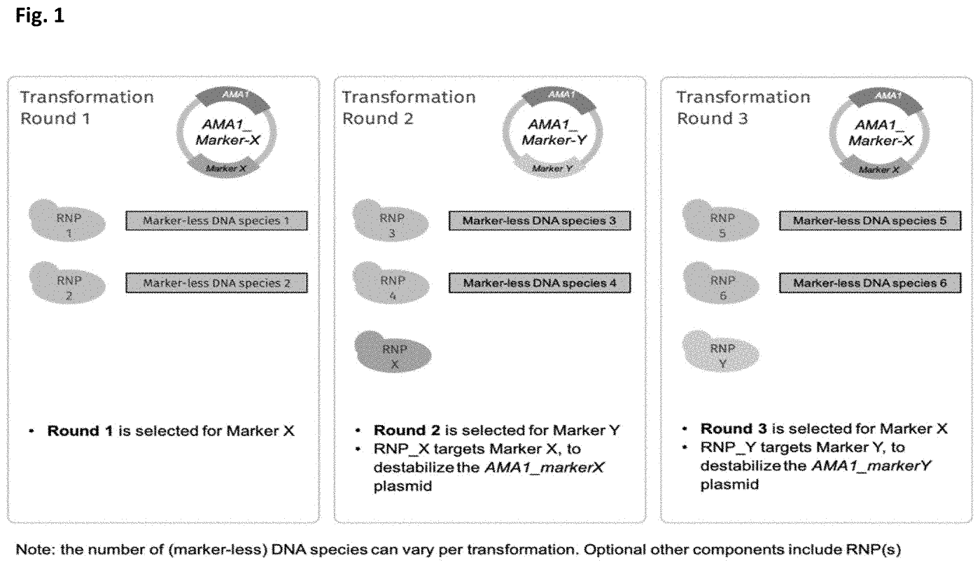

In some embodiments, the method of recycling an extra-chromosomally replicating plasmid comprises administering a RNP (FIG. 1). In some embodiments, the RNP comprises a gRNA and a nuclease, wherein the gRNA recognizes a site on the extra-chromosomally replicating plasmid and directs cleavage of a target nucleic acid by a nuclease. In some embodiments, the target nucleic acid is a selectable marker gene on the extra-chromosomally replicating plasmid or fragment thereof. In some embodiments, the target nucleic acid is a nucleic acid within 500 base pairs of the selectable marker gene. In some embodiments, the target nucleic acid is about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 nucleotides of a selectable marker gene. In some embodiments, addition of an RNP cleaves and destabilizes the extra-chromosomally replicating plasmid, facilitating its removal from a competent cell.

Non-limiting examples of nucleases include nucleases with at least about 80%, at least about 85%, at least about 90%, at least about 95%, at least about 96%, at least about 97%, at least about 98%, at least about 99%, or about 100% identity to a nuclease is selected from the group consisting of Cas9, Cas12a (Cpf1), Cas12b. Cas12c, Cas12d, Cas12e, Cas12h, Tnp-B like, Cas13a (C2c2), Cas13b, Cas13c, Cpf1, Cas14, and MAD7, or homologs, orthologs, or paralogs thereof.

Non-limiting examples of gRNAs include single-molecule guide RNA (sgRNA) or dual-molecule guide RNA, e.g, RNA which comprises a crRNA and tracrRNA.

### Recycling of Extra-Chromosomally Replicating Plasmid Through Introduction of a Recombinase

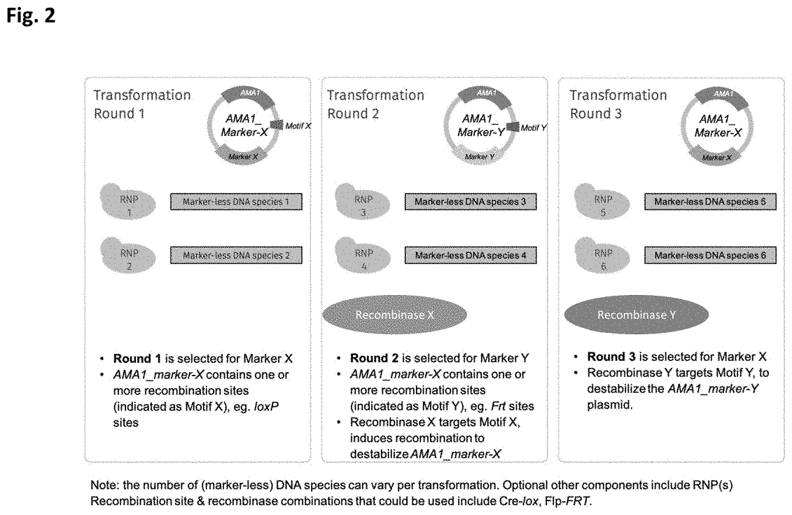

In some embodiments, the method of recycling an extra-chromosomally replicating plasmid comprises administering a recombinase or an integrase (FIG. 2). In some embodiments, a recombinase recognizes a recombinatorial site on the extra-chromosomally replicating plasmid. A recombinase catalyzes recombination of DNA between recombinatorial sites in a DNA molecule. In some embodiments, recombination destabilizes the extra-chromosomally replicating plasmid and facilitates its removal from a competent cell.

Non-limiting examples of recombinases and/or integrases include Cre recombinase, %-integrase, XerC recombinase, XerD recombinase, flippase (Flp), Flp recombinase, Hin recombinase, Tre recombinase, RecA recombinase, Rad51 recombinase, gamma-delta resolvase, and Dmc1 recombinase.

In some embodiments, the recombinatorial site is a loxP, a Frt, psi, dif, cer, attB, attP, attL, attR, att1, att2, or att site, or mutant, variant, or derivative thereof. In some embodiments, the recombinatorial site is recognized by a recombinase or integrase selected from the group of Cre recombinase, λ-integrase, XerC recombinase, XerD recombinase, flippase (Flp), Flp recombinase, Hin recombinase, Tre recombinase, RecA recombinase, Rad51 recombinase, gamma-delta resolvase, and Dmc1 recombinase. In some embodiments, the recombinatorial site is a loxP or a Frt site.

### Recycling of Extra-Chromosomally Replicating Plasmid Through Introduction of an Endonuclease

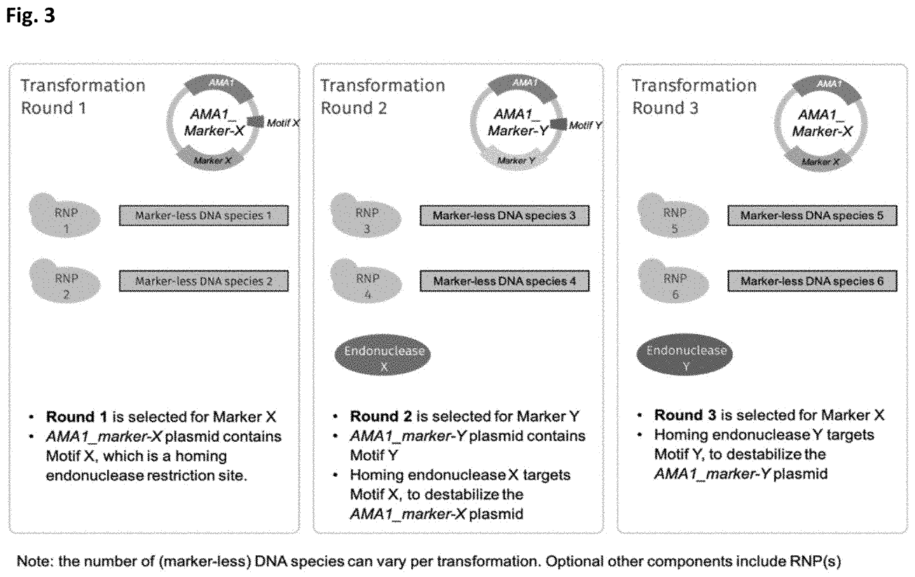

In some embodiments, the method of recycling an extra-chromosomally replicating plasmid comprises administering an endonuclease (FIG. 3). In some embodiments, an endonuclease recognizes a restriction site on the extra-chromosomally replicating plasmid. In some embodiments, the extra-chromosomally replicating plasmid comprises about 1, 2, 3, 4, 5, 6, 7, 8, 9, 10, or more restriction sites. One or more endonucleases cut DNA (on the extra-chromosomally replicating plasmid) at one or more restriction sites, producing discrete DNA fragments or producing linear DNA from plasmid DNA consequently destabilizing the extra-chromosomally replicating plasmid and encouraging its loss from the cell or mycelium.

Non-limiting examples of restriction sites and endonucleases include AatII, AbaSI, Acc65I, AccI, AciI, AclI, AcuI, AfeI, AflII, AflIII, AgeI, AhdI, AleI-v2, AluI, AlwI, AlwNI, ApaI, ApaLI, ApeKI, ApoI, AscI, AseI, AsiSI, AvaI, AvalI, AvrII, BaeGI, BaeI, BamHI, BanI, BanII, BbsI, BbvCI, BbvI, BccI, BceAI, BcgI, BciVI, BclI, BcoDI, BfaI, BfuAI, BglI, BglII, BlpI, BmgBI, BmrI, BmtI, BpmI, Bpu10I, BpuEI, BsaAI, BsaBI, BsaHI, BsaI, BsaJI, BsaWI, BsaXI, BseRI, BseYI, BsgI, BsiEI, BsiHKAI, BsiWI, BslI, BsmAI, BsmBI, BsmBI-v2, BsmFI, BsmI, BsoBI, Bsp1286I, BspCNI, BspDI, BspEI, BspHI, BspMI, BspQI, BsrBI, BsrDI, BsrFαI, BsrGI, BsrI, BssHII, BssSaI, BstAPI, BstBI, BstEII, BstNI, BstUI, BstXI, BstYI, Bsu36I, BtgI, BtgZI, BtsCI, BtsIMutI, BtsαI, Cac8I, ClaI, CspCI, CviAII, CviKI-1, CviQI, DdeI, DpnI, DpnII, DraI, DraIII, DrdI, EaeI, EagI, EarI, EciI, Eco53kI, EcoNI, EcoO109I, EcoRI, EcoRV, Esp3I, FatI, FauI, Fnu4HI, FokI, FseI, FspEI, FspI, HaeII, HaeIII, HgaI, HhaI, HincII, HindIII, HinfI, HinPII, HpaI, HpaII, HphI, Hpy166II, Hpy188I, Hpy188III, Hpy99I, HpyAV, HpyCH4III, HpyCH4IV, HpyCH4V, I-CeuI, I-SceI, KasI, KpnI, LpnPI, MboI, MboII, MfeI, MIuCI, MluI, MlyI, MmeI, MnlI, MscI, MseI, MslI, MspAlI, MspI, MspJI, MwoI, NaeI, NarI, Nb.BbvCI, Nb.BsmI, Nb.BsrDI, Nb.BssSI, Nb.BtsI, NciI, NcoI, NdeI, NgoMIV, NheI, NlaIII, NlaIV, NmeAIII, NotI, NruI, NsiI, NspI, Nt.AlwI, Nt.BbvCI, Nt.BsmAI, Nt.BspQI, Nt.BstNBI, Nt.CviPII, Pac, PaeR7I, PciI, PflFI, PflMI, PI-PspI, PI-SceI, PleI, PluTI, PmeI, PmlI, PpuMI, PshAI, PsiI, PsiI-v2, PspGI, PspOMI, PspXI, PstI, PvuI, PvuII, RsaI, RsrII, SacI, SacII, SalI, SapI, Sau3AI, Sau96I, SbfI, ScaI, ScrFI, SexAI, SfaNI, SfcI, SfiI, SfoI, SgrAI, SmaI, SmlI, SnaBI, SpeI, SpeI-HF®, SphI, SrfI, SspI, StuI, StyD41, StyI, SwaI, TaqαI, TfiI, TseI, Tsp451, TspMI, TspRI, Tth111I, XbaI, XcmI, XhoI, XmaI, XmnI, and ZraI.

### Recycling of Extra-Chromosomally Replicating Plasmid Through Induction of a Suicide Gene

In some embodiments, the method of recycling an extra-chromosomally replicating plasmid comprises removing the extra-chromosomally replicating plasmid via induction of a suicide gene which is under control of an inducible promoter. In some embodiments, the extra-chromosomally replicating plasmid comprises a suicide gene. Application of a reagent that induces expression of a suicide gene causes competent cells comprising the suicide gene to die.

In some embodiments, the suicide gene is under the control of an inducible promoter selected from the group consisting of an alcohol-regulated promoter, a tetracycline-regulated promoter, a steroid regulated promoter, a metal-regulated promoter, a pathogenesis regulated promoter, a heat shock promoter, a synthetic-transcription factor-dependent promoter, a carbon-regulated promoter, or a light-regulated promoter.

Non-limiting examples of a reagent that induces expression of a suicide gene include alcohol, an antibiotic, tetracycline, a steroid, a metal, a transcription factor, heat, light, xylose, glucose, sucrose, maltose, ethanol, glycerol, methanol, oleic acid, acetate, hexose, lactose, and galactose.

### IV. Methods for Genome Editing

In some embodiments, the disclosure provides methods for gene editing. In some embodiments, the methods described herein allow for the introduction of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, or more gene edits.

In some embodiments, the method comprises transforming a competent cell with a first composition comprising:

(a) an extra-chromosomally replicating plasmid, comprising a selectable marker gene; and

(b) a gene-editing complex that recognizes a genomic target of a competent cell. In some embodiments, the first composition comprises a genetic element of interest. In some embodiments, the first composition does not comprise a genetic element of interest. In some embodiments, the first composition comprises the extra-chromosomally replicating plasmid and a genetic element of interest.

In some embodiments, the extra-chromosomally replicating plasmid is the genetic element of interest. In some embodiments, the genetic element of interest is double stranded DNA. In some embodiments, the genetic element of interest is single stranded DNA. In some embodiments, the genetic element of interest is a plasmid. Various examples of genetic elements of interest are described in Section II of this disclosure. In some embodiments, the genetic element of interest is selected from the group consisting of: a nucleic acid sequence, a gene of interest, a gene variant, a genetic edit, a single nucleotide polymorphism, a genetic regulatory sequence, a promoter, a non-coding nucleic acid sequence, a terminator, or any combination thereof.

Upon transformation of the competent cell with the first composition, the RNP cleaves the genomic target from the competent cell via a CRISPR mechanism, and the genetic element of interest replaces the genomic target DNA. The following patent documents describe CRISPR and are incorporated by reference herein in their entirety: U.S. Pat. Nos. 8,697,359, 8,771,945, 8,795,965, 8,865,406, 8,871,445, 8,889,356, 8,895,308, 8,906,616, 8,932,814, 8,945,839, 8,993,233, 8,999,641, U.S. patent application Ser. No. 14/704,551, and U.S. patent application Ser. No. 13/842,859

Various methods for transformation are taught herein. In some embodiments, transformation of a competent cell involves heat-shock or electroporation. In some embodiments, transformation is automated. In some embodiments, competent cells are transformed using high-throughput electroporation systems, for example, the VWR®High-throughput Electroporation Systems, BTX™, Bio-Rad®, Gene Pulser MXcell™, or other multi-well electroporation systems. In some embodiments, transformation is mediated by polyethylene glycol (PEG).

In some embodiments, about 0.01 μg to about 100 μg of DNA, for example, about 0.01 μg, about 0.05 μg, about 0.1 μg, about 0.15 μg, about 0.2 μg, about 0.25 μg, about 0.3 μg, about 0.35 μg, about 0.4 μg, about 0.45 μg, about 0.5 μg, about 0.55 μg, about 0.6 μg, about 0.65 μg, about 0.7 μg, about 0.75 μg, about 0.8 μg, about 0.85 μg, about 0.9 μg, about 0.95 μg, about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, or about 100 μg of DNA (e.g. an extra-chromosomally replicating plasmid) is used to transform a competent cell.

In some embodiments, the method for gene editing comprises selecting for competent cells that comprise the extra-chromosomally replicating plasmid (e.g. transformed competent cells). In some embodiments, competent cells that comprise the extra-chromosomally replicating plasmid are selected by applying a selective agent to the competent cells.

Non-limiting examples of selective agents include antibiotics, such as ampicillin, tetracycline, zeocin, spectinomycin, kanamycin, neomycin, vancomycin, methicillin, oxacillin, erythromycin, linezolid, puromycin, and hygromycin. Non-limiting examples of selectable marker genes include pyrG, hph, nat, amdS, nptII, niaD, and argB.

In some embodiments, the selectable marker gene is an antibiotic resistance gene, for example, a chloramphenicol resistance gene, an ampicillin resistance gene, a tetracycline resistance gene, a Zeocin resistance gene, a spectinomycin resistance gene and a Km (Kanamycin resistance gene), tetA (tetracycline resistance gene), G418 (neomycin resistance gene), van (vancomycin resistance gene), methicillin (methicillin resistance gene), penicillin (penicillin resistance gene), oxacillin (oxacillin resistance gene), erythromycin (erythromycin resistance gene), linezolid (linezolid resistance gene), puromycin (puromycin resistance gene) or a hygromycin (hygromycin resistance gene).

Competent cells, extra-chromosomally replicating plasmids, genetic elements of interest, and gene-editing complexes that recognize a genomic target of a competent cell are described in Sections II and III of this disclosure. In some embodiments, the competent cell is a eukaryotic cell, a prokaryotic cell, a filamentous fungal cell, or a protoplast. In some embodiments, the extra-chromosomally replicating plasmid comprises a selectable marker gene, recombinatorial site, endonuclease site, suicide gene controlled by an inducible promoter, or combination thereof. In some embodiments, the gene-editing complex comprises a ribonucleoprotein (RNP), a TALEN, or a ZFN. In some embodiments, the first composition can be used to make between about 1 and about 100 genetic edits, for example about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 genetic edits.

In some embodiments, the first composition comprises between about 1 and about 100 gene-editing complexes (e.g., an RNP, ZFN, or TALEN), for example, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 gene-editing complexes. In some embodiments, one or more gene-editing complexes recognize different genomic targets. In some embodiments, one or more gene-editing complexes recognize the same genomic target.

In some embodiments, the first composition comprises between about 1 and about 100,000 genetic elements of interest, for example, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, about 700, about 710, about 720, about 730, about 740, about 750, about 760, about 770, about 780, about 790, about 800, about 810, about 820, about 830, about 840, about 850, about 860, about 870, about 880, about 890, about 900, about 910, about 920, about 930, about 940, about 950, about 960, about 970, about 980, about 990, about 1000, about 2000, about 3000, about 4000, about 5000, about 6000, about 7000, about 8000, about 9000, about 10000, about 11000, about 12000, about 13000, about 14000, about 15000, about 16000, about 17000, about 18000, about 19000, about 20000, about 21000, about 22000, about 23000, about 24000, about 25000, about 26000, about 27000, about 28000, about 29000, about 30000, about 31000, about 32000, about 33000, about 34000, about 35000, about 36000, about 37000, about 38000, about 39000, about 40000, about 41000, about 42000, about 43000, about 44000, about 45000, about 46000, about 47000, about 48000, about 49000, about 50000, about 51000, about 52000, about 53000, about 54000, about 55000, about 56000, about 57000, about 58000, about 59000, about 60000, about 61000, about 62000, about 63000, about 64000, about 65000, about 66000, about 67000, about 68000, about 69000, about 70000, about 71000, about 72000, about 73000, about 74000, about 75000, about 76000, about 77000, about 78000, about 79000, about 80000, about 81000, about 82000, about 83000, about 84000, about 85000, about 86000, about 87000, about 88000, about 89000, about 90000, about 91000, about 92000, about 93000, about 94000, about 95000, about 96000, about 97000, about 98000, about 99000, or about 100000 genetic elements of interest. In some embodiments, a genetic element of interest replaces one or more genomic targets. In some embodiments, two or more genetic elements of interest can replace the same genomic target. In some embodiments, two or more genetic elements of interest replace different genomic targets. In some embodiments, this gene editing method is utilized to produce a library of cells.

In some embodiments, the first composition comprises no genetic elements of interest.

In some embodiments, after RNP cleavage of the genomic target, genomic DNA is repaired by non-homologous end joining, homologous recombination, or micro-homology mediated repair.

In some embodiments, after selecting for cells that comprise a gene edit, an extra-chromosomally replicating plasmid is recycled according to the methods described in Section III of this disclosure.

In some embodiments, between 1 and 1000 additional rounds of gene editing is performed, for example, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, about 500, about 505, about 510, about 515, about 520, about 525, about 530, about 535, about 540, about 545, about 550, about 555, about 560, about 565, about 570, about 575, about 580, about 585, about 590, about 595, about 600, about 605, about 610, about 615, about 620, about 625, about 630, about 635, about 640, about 645, about 650, about 655, about 660, about 665, about 670, about 675, about 680, about 685, about 690, about 695, about 700, about 705, about 710, about 715, about 720, about 725, about 730, about 735, about 740, about 745, about 750, about 755, about 760, about 765, about 770, about 775, about 780, about 785, about 790, about 795, about 800, about 805, about 810, about 815, about 820, about 825, about 830, about 835, about 840, about 845, about 850, about 855, about 860, about 865, about 870, about 875, about 880, about 885, about 890, about 895, about 900, about 905, about 910, about 915, about 920, about 925, about 930, about 935, about 940, about 945, about 950, about 955, about 960, about 965, about 970, about 975, about 980, about 985, about 990, about 995, or about 1000 additional rounds.

In some embodiments, before each additional round, an extra-chromosomally replicating plasmid from a previous round is removed. Removal methods are described in Section III of this disclosure.

In some embodiments, each round of gene editing is numbered according to the order in which it is performed. For example, the first additional round is called the second round, and the second additional round is called the third round and so on.

In some embodiments, each additional round comprises transforming a competent cell with an additional composition. In some embodiments, each additional composition is numbered according to its round number. For example, the composition of the first additional round (e.g. the second round) is the second composition, and the composition of the second additional round (e.g. the third round) is the third composition and so on. Each additional composition comprises:

(a) a second extra-chromosomally replicating plasmid, wherein the extra-chromosomally replicating plasmid comprises a second selectable marker gene; and

(b) a gene-editing complex that recognizes a genomic target of a competent cell.

Characteristics of the second extra-chromosomally replicating plasmid and gene-editing complex are described in Section II of this disclosure.

In some embodiments, extra-chromosomally replicating plasmids of different rounds comprise the same selectable marker gene. In some embodiments, extra-chromosomally replicating plasmids of different rounds comprise different selectable marker genes.

In some embodiments, extra-chromosomally replicating plasmids comprise one or more of a recombinatorial site, a suicide gene, or a endonuclease site, as described in Sections II and III of this disclosure. In some embodiments, extra-chromosomally replicating plasmids from different rounds comprise different combinations of a recombinatorial site, a suicide gene, or an endonuclease site. In some embodiments, extra-chromosomally replicating plasmids from different rounds comprise the same combinations of a recombinatorial site, a suicide gene, or an endonuclease site.

### EXAMPLES

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1. Use of RNPs to Recycle Extra-Chromosomally Replicating Plasmids for Gene-Editing

Fungal protoplasts are transformed with a composition comprising:

(a) an extra-chromosomally replicating plasmid, wherein the extra-chromosomally replicating plasmid comprises a selectable marker gene;

(b) At least one gene-editing complex that recognizes at least one genomic target of the competent cell; and

(c) optionally 1-100,000 genetic elements of interest.

Protoplasts that comprise the composition are selected for by employing a selective agent. Protoplasts that grow in the presence of a selective agent have been transformed. The fungal protoplasts are subsequently transformed with a second composition. The second composition enables removal of the first extra-chromosomally replicating plasmid via a RNP. The second composition comprises:

(a) a second extra-chromosomally replicating plasmid, wherein the extra-chromosomally replicating plasmid comprises a selectable marker gene;

(b) At least one gene-editing complex that recognizes at least one genomic target of the competent cell, and

(c) optionally 1-100,000 genetic elements of interest;

(d) an RNP that comprises Cas9 and a gRNA that recognizes the selectable marker gene of round 1 to remove the extra-chromosomally replicating plasmid utilized during round 1.

Protoplasts that grow in the presence of a selective agent have been transformed.

The fungal protoplasts are subsequently transformed with a third composition. The third composition enables removal of the second extra-chromosomally replicating plasmid via a RNP. The third composition comprises:

(a) a third extra-chromosomally replicating plasmid, wherein the extra-chromosomally replicating plasmid comprises a selectable marker gene;

(b) At least one gene-editing complex that recognizes at least one genomic target of the competent cell, and

(c) optionally 1-100,000 genetic elements of interest;

(d) an RNP that comprises Cas9 and a gRNA that recognizes the selectable marker gene of round 2 to remove the extra-chromosomally replicating plasmid utilized during round 2 (FIG. 1).

### Example 2. Use of Recombinase to Recycle Extra-Chromosomally Replicating Plasmids for Gene-Editing

Fungal protoplasts are transformed with a composition comprising:

(a) An extra-chromosomally replicating plasmid, wherein the extra-chromosomally replicating plasmid comprises a selectable marker gene and at least two recombinatorial sites;

(b) At least one gene-editing complex that recognizes at least one genomic target of the competent cell; and

(c) optionally 1-100,000 genetic elements of interest.

Protoplasts that comprise the composition are selected for by employing a selective agent. Protoplasts that grow in the presence of a selective agent have been transformed. The fungal protoplasts are transformed with a second composition. The second composition enables removal of the first extra-chromosomally replicating plasmid via a recombinase. The second composition comprises:

(a) a second extra-chromosomally replicating plasmid, wherein the extra-chromosomally replicating plasmid comprises a selectable marker gene;

(b) at least one gene-editing complex that recognizes at least one genomic target of the competent cell; and

(c) optionally 1-100,000 genetic elements of interest;

(d) a recombinase that recognizes a recombinatorial site (e.g., Motif X) on the extra-chromosomally replicating plasmid.

Protoplasts that grow in the presence of a selective agent have been transformed. The fungal protoplasts are transformed with a third composition. The third composition enables removal of the second extra-chromosomally replicating plasmid via a recombinase. The third composition comprises:

(a) a third extra-chromosomally replicating plasmid, wherein the extra-chromosomally replicating plasmid comprises a selectable marker gene.

(b) at least one gene-editing complex that recognizes at least one genomic target of the competent cell; and

(c) optionally 1-100,000 genetic elements of interest;

(d) a recombinase that recognizes a recombinatorial site (e.g., Motif Y) on the extra-chromosomally replicating plasmid of round 2. (FIG. 2).

### Example 3. Use of Endonuclease to Recycle Extra-Chromosomally Replicating Plasmids for Gene-Editing

Fungal protoplasts are transformed with a composition comprising:

(a) an extra-chromosomally replicating plasmid, wherein the extra-chromosomally replicating plasmid comprises a selectable marker gene;

(b) At least one gene-editing complex that recognizes at least one genomic target of the competent cell; and

(c) optionally 1-100,000 genetic elements of interest.

Protoplasts that comprise the composition are selected for by employing a selective agent. Protoplasts that grow in the presence of a selective agent have been transformed. The fungal protoplasts are transformed with a second composition. The second composition enables removal of the first extra-chromosomally replicating plasmid via a restriction endonuclease. The second composition comprises:

(a) a second extra-chromosomally replicating plasmid, wherein the extra-chromosomally replicating plasmid comprises a selectable marker gene;

(b) At least one gene-editing complex that recognizes at least one genomic target of the competent cell; and

(c) optionally 1-100,000 genetic elements of interest; and

(d) a restriction endonuclease (e.g. a homing endonuclease) that recognizes the endonuclease site (e.g., Motif X) on the extra-chromosomally replicating plasmid of round 1.

(e) Protoplasts that grow in the presence of a selective agent have been transformed. The fungal protoplasts are transformed with a third composition. The third composition enables removal of the second extra-chromosomally replicating plasmid via a restriction endonuclease. The third composition comprises.

(f) a third extra-chromosomally replicating plasmid, wherein the extra-chromosomally replicating plasmid comprises a selectable marker gene;

(g) at least one gene-editing complex that recognizes at least one genomic target of the competent cell; and

(h) optionally 1-100,000 genetic elements of interest; and

(i) a restriction endonuclease (e.g. a homing endonuclease) that recognizes the endonuclease site (e.g., Motif Y) on the extra-chromosomally replicating plasmid of round 2 (FIG. 3).

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

### Additional Embodiments of the Disclosure

The following embodiments are also envisioned by the present disclosure:

1. A composition for gene editing, comprising:

(a) competent cells:

(b) an extra-chromosomally replicating plasmid comprising a selectable marker gene; and

(c) a gene-editing complex that recognizes a genomic target of a competent cell.

2. The composition of embodiment 1, comprising a genetic element of interest.
3. The composition of embodiment 1, wherein the composition does not contain a genetic element of interest.
4. The composition of embodiment 2, wherein the genetic element of interest is selected from the group consisting of: a nucleic acid sequence, a gene of interest, a gene variant, a genetic edit, a single nucleotide polymorphism, a genetic regulatory sequence, a promoter, a non-coding nucleic acid sequence, a terminator, or any combination thereof.
5. The composition of embodiment 2, wherein the genetic element of interest is a promoter.
6. The composition of embodiment 2, wherein the genetic element of interest is a gene or fragment thereof.
7. The composition of any one of embodiments 1-6, wherein the gene-editing complex comprises a ribonucleoprotein (RNP).
8. The composition of embodiment 7, wherein the RNP comprises Cas9 and a guide RNA (gRNA) that recognizes the genomic target.
9. The composition of any one of embodiments 1-6, wherein the gene-editing complex comprises a transcription activator-like effector nuclease (TALEN).
10. The composition of any one of embodiments 1-6, wherein the gene-editing complex comprises a zinc-finger nuclease (ZFN).
11. The composition of any one of embodiments 1-10, wherein the competent cells are eukaryotic cells.
12. The composition of any one of embodiments 1-10, wherein the competent cells are prokaryotic cells.
13. The composition of any one of embodiments 1-10, wherein the competent cells are fungal cells.
14. The composition of any one of embodiments 1-10 or 13, wherein the competent cells are filamentous fungal cells.
15. The composition of any one of embodiments 1-10, 13, or 14, wherein the competent cells are protoplasts.
16. The composition of any one of embodiments 1-15, wherein the extra-chromosomally replicating plasmid comprises a plasmid replicator.
17. The composition of embodiment 16, wherein the plasmid replicator is AMA1.
18. The composition of any one of embodiments 1-17, wherein the selectable marker gene is selected from pyrG, hph, nat, amdS, nptII, niaD, and argB.
19. The composition of any one of embodiments 1-18, wherein the extra-chromosomally replicating plasmid comprises an endonuclease site.
20. The composition of any one of embodiments 1-19, wherein the extra-chromosomally replicating plasmid comprises a recombinatorial site.
21. The composition of embodiment 20, wherein the recombinatorial site is a loxP site or a Frt site.
22. The composition of any one of embodiments 1-21, comprising a RNP that recognizes the selectable marker gene.
23. The composition of embodiment 22, wherein the RNP comprises Cas9 and a gRNA.
24. The composition of any one of embodiments 1-23, comprising an endonuclease, which recognizes an endonuclease site.
25. The composition of any one of embodiments 1-14, comprising a recombinase, which recognizes a recombinatorial site.
26. The composition of any one of embodiments 1-25, wherein the extra-chromosomally replicating plasmid comprises a suicide gene, wherein the suicide gene is under control of an inducible promoter.
27. The composition of embodiment 26, wherein the inducible promoter is an alcohol-regulated promoter, a tetracycline-regulated promoter, a steroid regulated promoter, a metal-regulated promoter, a pathogenesis regulated promoter, a carbon-regulated promoter, a xylose-regulated promoter, a heat shock promoter, a synthetic-transcription factor-dependent promoter, or a light-regulated promoter.
28. The composition of embodiment 26, wherein expression of the suicide gene is induced by an alcohol, a transcription factor, tetracycline, a steroid, a metal, heat, light, an antibiotic, a sugar, xylose, glucose, sucrose, maltose, ethanol, glycerol, methanol, oleic acid, acetate, hexose, lactose, or galactose.
29. A method for gene editing, comprising: transforming a competent cell with a first composition comprising:

(a) an extra-chromosomally replicating plasmid comprising a selectable marker gene; and

(b) a gene-editing complex that recognizes a genomic target of a competent cell.

30. The method of embodiment 29, comprising a genetic element of interest.
31. The method of embodiment 29, wherein the composition does not contain a genetic element of interest.
32. The method of embodiment 30, wherein the genetic element of interest is selected from the group consisting of: a nucleic acid sequence, a gene of interest, a gene variant, a genetic edit, a single nucleotide polymorphism, a genetic regulatory sequence, a promoter, a non-coding nucleic acid sequence, a terminator, or any combination thereof.
33. The method of embodiment 30, wherein the genetic element of interest is a promoter.
34. The method of embodiment 30, wherein the genetic element of interest is a gene or fragment thereof.
35. The method of any one of embodiments 29-34, wherein the gene-editing complex comprises a ribonucleoprotein (RNP).
36. The method of embodiment 29, wherein the RNP comprises Cas9 and a guide RNA (gRNA) that recognizes the genomic target.
37. The method of any one of embodiments 29-36, wherein the gene-editing complex comprises a transcription activator-like effector nuclease (TALEN).
38. The method of any one of embodiments 29-37, wherein the gene-editing complex comprises a zinc-finger nuclease (ZFN).
39. The method of any one of embodiments 29-38, comprising selecting for competent cells that comprise the extra-chromosomally replicating plasmid.
40. The method of any one of embodiments 29-39, wherein the extra-chromosomally replicating plasmid comprises a plasmid replicator.
41. The method of embodiment 40, wherein the plasmid replicator is AMA1.
42. The method of any one of embodiments 29-41, wherein the selectable marker gene is selected from pyrG, hph, nat, amdS, nptII, niaD, and argB.
43. The method of any one of embodiments 29-42, wherein the extra-chromosomally replicating plasmid comprises a endonuclease site.
44. The method of any one of embodiments 29-43, wherein the extra-chromosomally replicating plasmid comprises a recombinatorial site.
45. The method of embodiment 44, wherein the recombinatorial site is a loxP site or a Fri site.
46. The method of any one of embodiments 29-45, comprising removing the extra-chromosomally replicating plasmid.
47. The method of any one of embodiments 29-46, comprising removing the extra-chromosomally replicating plasmid by applying a RNP to the competent cells comprising the extra-chromosomally replicating plasmid.
48. The method of embodiment 47, wherein the RNP comprises Cas9 and a gRNA that recognizes the selectable marker gene of the extra-chromosomally replicating plasmid.
49. The method of any one of embodiments 29-46, comprising removing the extra-chromosomally replicating plasmid by applying a recombinase to the competent cells comprising the extra-chromosomally replicating plasmid, wherein the recombinase recognizes a recombinatorial site on the extra-chromosomally replicating plasmid.
50. The method of any one of embodiments 29-42, comprising removing the extra-chromosomally replicating plasmid by applying an endonuclease to the competent cells comprising the extra-chromosomally replicating plasmid, wherein the endonuclease recognizes an endonuclease site on the extra-chromosomally replicating plasmid.
51. The method of any one of embodiments 29-50, wherein the genetic element of interest is introduced at the genomic target site.
52. The method of any one of embodiments 29-51, wherein the extra-chromosomally replicating plasmid comprises a suicide gene, wherein the suicide gene is under control of an inducible promoter.
53. The method of embodiment 52, wherein the inducible promoter is an alcohol-regulated promoter, a tetracycline-regulated promoter, a steroid regulated promoter, a metal-regulated promoter, a pathogenesis regulated promoter, a heat shock promoter, a carbon-regulated promoter, a xylose-regulated promoter, a synthetic-transcription factor-dependent promoter or a light-regulated promoter.
54. The method of any one of embodiments 29-53, comprising removing the extra-chromosomally replicating plasmid by inducing the promoter to express the suicide gene.
55. The method of embodiment 54, wherein inducing comprises introducing an alcohol, a transcription factor, tetracycline, a steroid, a metal, heat, light, an antibiotic, a sugar, xylose, glucose, sucrose, maltose, ethanol, glycerol, methanol, oleic acid, acetate, hexose, lactose, or galactose to the competent cells comprising the extra-chromosomally replicating plasmid.
56. The method of any one of embodiments 29-55, wherein the competent cell is a eukaryotic cell.
57. The method of any one of embodiments 29-56, wherein the competent cell is a prokaryotic cell.
58. The method of any one of embodiments 29-57, wherein the competent cell is a fungal cell.
59. The method of any one of embodiments 29-58, wherein the competent cell is a filamentous fungal cell.
60. The method of any one of embodiments 29-59, wherein the competent cell is a protoplast.
61. The method of any one of embodiments 29-60, comprising introducing a second composition, comprising:

(a) a second extra-chromosomally replicating plasmid, wherein the extra-chromosomally replicating plasmid comprises a second selectable marker gene; and

(b) a gene-editing complex that recognizes a genomic target of a competent cell.

62. The method of embodiment 61, wherein the second composition comprises a genetic element of interest.
63. The method of embodiment 61, wherein the second composition does not comprise a genetic element of interest.
64. The method of embodiment 62, wherein the genetic element of interest of the second composition is selected from the group consisting of: a nucleic acid sequence, a gene of interest, a gene variant, a genetic edit, a single nucleotide polymorphism, a genetic regulatory sequence, a promoter, a non-coding nucleic acid sequence, a terminator, or any combination thereof.
65. The method of embodiment 62, wherein the genetic element of interest of the second composition is a promoter.
66. The method of embodiment 62, wherein the genetic element of interest of the second composition is a gene or fragment thereof.
67. The method of any one of embodiments 61-66, wherein the gene-editing complex comprises a ribonucleoprotein (RNP).
68. The method of embodiment 67, wherein the RNP comprises Cas9 and a guide RNA (gRNA) that recognizes the genomic target.
69. The method of any one of embodiments 61-66, wherein the gene-editing complex comprises a transcription activator-like effector nuclease (TALEN).
70. The method of any one of embodiments 61-66, wherein the gene-editing complex comprises a zinc-finger nuclease (ZFN).
71. The method of any one of embodiments 61-70, comprising selecting for competent cells that comprise the second extra-chromosomally replicating plasmid.
72. The method of any one of embodiments 61-71, wherein the second extra-chromosomally replicating plasmid comprises a plasmid replicator.
73. The method of embodiment 72, wherein the plasmid replicator is AMA1.
74. The method of any one of embodiments 61-73, wherein the second selectable marker gene is selected from pyrG, hph, nat, amdS, nptII, niaD, and argB.
75. The method of any one of embodiments 61-74, wherein the second extra-chromosomally replicating plasmid comprises a endonuclease site.
76. The method of any one of embodiments 61-74, wherein the second extra-chromosomally replicating plasmid comprises a recombinatorial site.
77. The method of embodiment 76, wherein the recombinatorial site is a loxP site or a Frt site.
78. The method of any one of embodiments 61-77, comprising removing the second extra-chromosomally replicating plasmid.
79. The method of any one of embodiments 61-78, comprising removing the second extra-chromosomally replicating plasmid by applying a ribonucleoprotein (RNP) to the competent cells comprising the second extra-chromosomally replicating plasmid.
80. The method of embodiment 79, wherein the RNP comprises Cas9 and a gRNA that recognizes the selectable marker gene of the second extra-chromosomally replicating plasmid.
81. The method of any one of embodiments 61-78, comprising removing the second extra-chromosomally replicating plasmid by applying a recombinase to the competent cells comprising the second extra-chromosomally replicating plasmid, wherein the recombinase recognizes a recombinatorial site on the second extra-chromosomally replicating plasmid.
82. The method of any one of embodiments 61-78, comprising removing the extra-chromosomally replicating plasmid by applying an endonuclease to the competent cells comprising the extra-chromosomally replicating plasmid, wherein the endonuclease recognizes an endonuclease site on the extra-chromosomally replicating plasmid.
83. The method of any one of embodiments 61-82, wherein the genetic element of interest of the second composition is introduced at a genomic target site of the second composition.
84. The method of any one of embodiments 61-83, wherein the extra-chromosomally replicating plasmid comprises a suicide gene, wherein the suicide gene is under control of an inducible promoter.
85. The method of embodiment 84, wherein the inducible promoter is an alcohol-regulated promoter, a tetracycline-regulated promoter, a steroid regulated promoter, a metal-regulated promoter, a pathogenesis regulated promoter, a heat shock promoter, a carbon-regulated promoter, a xylose-regulated promoter, a synthetic-transcription factor-dependent promoter, or a light-regulated promoter.
86. The method of embodiment 84 or 85, comprising removing the extra-chromosomally replicating plasmid by inducing the promoter to express the suicide gene.
87. The method of embodiment 86, wherein inducing comprises introducing an alcohol, a transcription factor, tetracycline, a steroid, a metal, heat, light, an antibiotic, a sugar, xylose, glucose, sucrose, maltose, ethanol, glycerol, methanol, oleic acid, acetate, hexose, lactose, or galactose to the competent cells comprising the second extra-chromosomally replicating plasmid.
88. The method of any one of embodiments 61-87, wherein the competent cell is a eukaryotic cell.
89. The method of any one of embodiments 61-87, wherein the competent cell is a prokaryotic cell.
90. The method of any one of embodiments 61-87, wherein the competent cell is a fungal cell.
91. The method of any one of embodiments 61-87 or 90, wherein the competent cell is a filamentous fungal cell.
92. The method of any one of embodiments 61-87, 90, or 91, wherein the competent cell is a protoplast.
93. A method of removing an extra-chromosomally replicating plasmid comprising a selectable marker gene from a competent cell, comprising: administering a reagent to remove the extra-chromosomally replicating plasmid.
94. The method of embodiment 93, wherein the extra-chromosomally replicating plasmid comprises a plasmid replicator.
95. The method of embodiment 94, wherein the plasmid replicator is AMA1.
96. The method of any one of embodiments 93-95, wherein the selectable marker gene is selected from pyrG, hph, nat, amdS, nptII, niaD, and argB.
97. The method of any one of embodiments 93-96, wherein the competent cell is a eukaryotic cell.
98. The method of any one of embodiments 93-96, wherein the competent cell is a prokaryotic cell.
99. The method of any one of embodiments 93-96, wherein the competent cell is a fungal cell.
100. The method of any one of embodiments 93-96 or 99, wherein the competent cell is a filamentous fungal cell.
101. The method of any one of embodiments 93-96, 99, or 100, wherein the competent cell is a protoplast.
102. The method of any one of embodiments 93-101, wherein the reagent comprises a ribonucleoprotein (RNP), an endonuclease, or a recombinase.
103. The method of any one of embodiments 93-102, wherein the reagent comprises a ribonucleoprotein (RNP) that recognizes the selectable marker gene.
104. The method of embodiment 103, wherein the RNP comprises Cas9 and a gRNA.
105. The method of any one of embodiments 93-104, wherein the extra-chromosomally replicating plasmid comprises an endonuclease site.
106. The method of embodiment 105, wherein the reagent is an endonuclease that recognizes an endonuclease site.
107. The method of any one of embodiments 93-106, wherein the extra-chromosomally replicating plasmid comprises a recombinatorial site.
108. The method of embodiment 107, wherein the recombinatorial site is a loxP site or a Fri site.
109. The method of any one of embodiments 93-108, wherein the reagent is a recombinase that recognizes a recombinatorial site.
110. The method of any one of embodiments 93-109, wherein the extra-chromosomally replicating plasmid comprises a suicide gene, wherein the suicide gene is under control of an inducible promoter.
111. The method of embodiment 110, wherein the inducible promoter is an alcohol-regulated promoter, a tetracycline-regulated promoter, a steroid regulated promoter, a metal-regulated promoter, a pathogenesis regulated promoter, a heat shock promoter, a carbon-regulated promoter, a xylose-regulated promoter, a synthetic-transcription factor-dependent promoter, or a light-regulated promoter.
112. The method of embodiment 110 or 111, comprising introducing a reagent to induce expression of the suicide gene, wherein the reagent is selected from the group consisting of a metal, a transcription factor, heat, light, an antibiotic, a sugar, xylose, glucose, sucrose, maltose, ethanol, glycerol, methanol, oleic acid, acetate, hexose, lactose, and galactose.
113. A method for making markerless multiple genomic edits, comprising:

(a) transforming a competent cell with a first composition comprising:
(i) an extra-chromosomally replicating plasmid comprising a selectable marker gene; and
(ii) a gene-editing complex that recognizes a genomic target of a competent cell;

(b) selecting for competent cells that comprise the extra-chromosomally replicating plasmid of the first composition;

(c) removing the extra-chromosomally replicating plasmid of the first composition by administering a reagent;

(d) transforming a competent cell with a second composition comprising:
(i) an extra-chromosomally replicating plasmid comprising a selectable marker gene; and
(ii) a gene-editing complex that recognizes a genomic target of a competent cell;

(e) selecting for competent cells that comprise the extra-chromosomally replicating plasmid of the second composition; and

(f) removing the extra-chromosomally replicating plasmid of the second composition by administering a reagent.

114. The method of embodiment 113, wherein the first composition comprises a genetic element of interest.
115. The method of embodiment 113 or 114, wherein the second composition comprises a genetic element of interest.
116. The method of any one of embodiments 113-115, comprising:

(a) transforming the competent cell with a third composition comprising:
(i) an extra-chromosomally replicating plasmid comprising a selectable marker gene; and
(ii) a gene-editing complex that recognizes a genomic target of a competent cell.

(b) selecting for competent cells that comprise the extra-chromosomally replicating plasmid of the third composition; and

(c) removing the extra-chromosomally replicating plasmid of the third composition by administering a reagent.

117. The method of embodiment any one of embodiments 113-116, wherein the first extra-chromosomally replicating plasmid is removed by administering a recombinase that recognizes a recombinatorial site on the first extra-chromosomally replicating plasmid.
118. The method of any one of embodiments 113-117, wherein the second extra-chromosomally replicating plasmid is removed by administering a recombinase that recognizes a recombinatorial site on the second extra-chromosomally replicating plasmid.
119. The method of any one of embodiments 113-118, wherein the first extra-chromosomally replicating plasmid is removed by administering an endonuclease that recognizes an endonuclease site on the first extra-chromosomally replicating plasmid.
120. The method of any one of embodiments 113-119, wherein the second extra-chromosomally replicating plasmid is removed by administering an endonuclease that recognizes an endonuclease site on the second extra-chromosomally replicating plasmid.
121. The method of any one of embodiments 113-120, wherein the first extra-chromosomally replicating plasmid is removed by administering a RNP that recognizes a selectable marker gene on the first extra-chromosomally replicating plasmid.
122. The method of any one of embodiments 113-121, wherein the second extra-chromosomally replicating plasmid is removed by administering a RNP that recognizes a selectable marker gene on the second extra-chromosomally replicating plasmid.
123. The method of embodiment 121, wherein the RNP comprises a gRNA and Cas9.
124. The method of embodiment 122, wherein the RNP comprises a gRNA and Cas9.
125. The method of any one of embodiments 113-124, wherein the first extra-chromosomally replicating plasmid is removed by administering an inducer of a suicide gene on the first extra-chromosomally replicating plasmid.
126. The method of embodiments 113-125, wherein the second extra-chromosomally replicating plasmid is removed by administering an inducer of a suicide gene on the second extra-chromosomally replicating plasmid.

What is claimed is:

1. A composition for gene editing, comprising:
(a) competent cells;
(b) an extra-chromosomally replicating plasmid comprising a selectable marker gene; and
(c) a gene-editing complex that recognizes a genomic target of a competent cell.

2. The composition of claim 1, comprising a genetic element of interest.

3. The composition of claim 1, wherein the composition does not contain a genetic element of interest.

4. The composition of claim 2, wherein the genetic element of interest is selected from the group consisting of: a nucleic acid sequence, a gene of interest, a gene variant, a genetic edit, a single nucleotide polymorphism, a genetic regulatory sequence, a promoter, a non-coding nucleic acid sequence, a terminator, or any combination thereof.

5. The composition of claim 2, wherein the genetic element of interest is a promoter or a gene or fragment thereof.

6. The composition of claim 1, wherein the gene-editing complex comprises a ribonucleoprotein (RNP).

7. The composition of claim 6, wherein the RNP comprises Cas9 and a guide RNA (gRNA) that recognizes the genomic target.

8. The composition of claim 1, wherein the gene-editing complex comprises a transcription activator-like effector nuclease (TALEN) or a zinc-finger nuclease (ZFN).

9. The composition of claim 1, wherein the competent cells are any one of eukaryotic cells, prokaryotic cells, fungal cells, filamentous fungal cells, or protoplasts.

10. The composition of claim 1, wherein the extra-chromosomally replicating plasmid comprises:
a) a plasmid replicator;
b) an endonuclease site; and/or
c) a recombinatorial site.

11. The composition of claim 10, wherein the plasmid replicator is AMA1.

12. The composition of claim 10, wherein the recombinatorial site is a loxP site or a Frt site.

13. The composition of claim 1, wherein the selectable marker gene is selected from pvrG, hph, nat, amdS, nptII, niaD, and argB.

14. The composition of claim 1, comprising a RNP that recognizes the selectable marker gene.

15. The composition of claim 14, wherein the RNP comprises Cas9 and a gRNA.

16. The composition of claim 1, comprising an endonuclease, which recognizes an endonuclease site, and/or a recombinase, which recognizes a recombinatorial site.

17. The composition of claim 1, wherein the extra-chromosomally replicating plasmid comprises a suicide gene, wherein the suicide gene is under control of an inducible promoter.

18. The composition of claim 17, wherein expression of the suicide gene:
a) is under control of an inducible promoter, and wherein the inducible promoter is an alcohol-regulated promoter, a tetracycline-regulated promoter, a steroid regulated promoter, a metal-regulated promoter, a pathogenesis regulated promoter, a carbon-regulated promoter, a xylose-regulated promoter, a heat shock promoter, a synthetic-transcription factor-dependent promoter, or a light-regulated promoter; and/or
b) is induced by an alcohol, a transcription factor, tetracycline, a steroid, a metal, heat, light, an antibiotic, a sugar, xylose, glucose, sucrose, maltose, ethanol, glycerol, methanol, oleic acid, acetate, hexose, lactose, or galactose.

19. A method for gene editing, comprising: transforming a competent cell with a first composition comprising:
(a) an extra-chromosomally replicating plasmid comprising a selectable marker gene; and
(b) a gene-editing complex that recognizes a genomic target of a competent cell.

20. A method of removing an extra-chromosomally replicating plasmid comprising a selectable marker gene from a competent cell, comprising: administering a reagent to remove the extra-chromosomally replicating plasmid.


SEQUENCE LISTING (8 sequences)

SEQ ID NO: 1 - DNA, 5749 bp, synthetic construct
  misc_feature 1..5749: note = AMA1 element
  source 1..5749: mol_type = other DNA
ccgggcccagtatatgttccgcagatgactggagctctgccatacgtgccctctcaagcaccatttgttccatctacagagactagtcaccaactagtctatcaagactcacagggtacattgctgagaccaactgaccagaggcagggtagcggattgacggctccatctccttcacttacaaggtctattgaaagccctttagcatcaccaagcggagaatagattgttaagcttattttttgtatactgttttgtgatagcacgaagtttttccacggtatcttgtaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgccataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgactcccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggaccattgtgcaatgtaaactccatgccgtgggagttcttgtccttcaagtgcttgaccaatatgtttctgttggcagagggaacctgtcaactagttaataactagtcagaaactatgatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgttatcgcgaatgctcggcatactaactgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaagagatttcgactagttatgtacctggagtttggatataggaatgtgttgtggtaacgaaatgtaagggggaggaaagaaaaagtcgtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtaccaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaacggtgagagtccagtggtcgcagataactccctaaattccctgagctaactctaagtcgaccatgccgtttatggttagcgcctcccaaaaaggaatggccgacttagagttacctcttgacgactttttctttcctcccccttacatttcgttaccacaacacattcctatatccaaactccaggtacataactagtcgaaatctctttaaatctagtcaagaactagatacttaaccttcatactgcttagagatagcctttgaagctcttatttcgctctttgtctcactctcaccttcctcctcctcctccaatcctttttgctcgggactagtccaagactagtcaacagttagtatgccgagcattcgcgataacgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatcatagtttctgactagttattaactagttgacaggttccctctgccaacagaaacatattggtcaagcacttgaaggacaagaactcccacggcatggagtttacattgcacaatggtcctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggagtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatggcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttacaagataccgtggaaaaacttcgtgctatcacaaaacagtatacaaaaaataagcttaacaatctattctccgcttggtgatgctaaagggctttcaatagaccttgtaagtgaaggagatggagccgtcaatccgctaccctgcctctggtcagttggtctcagcaatgtaccctgtgagtcttgatagactagttggtgactagtctctgtagatggaacaaatggtgcttgagagggcacgtatggcagagctccagtcatctgcggaacatatactgggcccgggaagatctcatggtcatagctgtttccg

SEQ ID NO: 2 - DNA, 3048 bp, synthetic construct
  misc_feature 1..3048: note = half AMA1 element
  source 1..3048: mol_type = other DNA
ccgggcccagtatatgttccgcagatgactggagctctgccatacgtgccctctcaagcaccatttgttccatctacagagactagtcaccaactagtctatcaagactcacagggtacattgctgagaccaactgaccagaggcagggtagcggattgacggctccatctccttcacttacaaggtctattgaaagccctttagcatcaccaagcggagaatagattgttaagcttattttttgtatactgttttgtgatagcacgaagtttttccacggtatcttgtaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgccataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgactcccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggaccattgtgcaatgtaaactccatgccgtgggagttcttgtccttcaagtgcttgaccaatatgtttctgttggcagagggaacctgtcaactagttaataactagtcagaaactatgatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgttatcgcgaatgctcggcatactaactgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaagagatttcgactagttatgtacctggagtttggatataggaatgtgttgtggtaacgaaatgtaagggggaggaaagaaaaagtcgtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtaccaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaacggtgagagtcca

SEQ ID NO: 3-8 - skipped (6 entries)